(19) United States
(12) Patent Application Publication
(10) Pub. No.: US 2024/0408396 A1
(43) Pub. Date: Dec. 12, 2024

(54) MULTI-NETWORK NEUROMODULATION

(71) Applicant: Dirk De Ridder, Dunedin (NZ)

(72) Inventor: Dirk De Ridder, Dunedin (NZ)

(21) Appl. No.: 18/656,558

(22) Filed: May 6, 2024

Related U.S. Application Data
(60) Provisional application No. 63/464,182, filed on May 4, 2023.

(51) Int. Cl. A61N 1/36

(52) U.S. Cl. CPC A61N 1/36171; A61N 1/36096; A61N 1/36178

(57) ABSTRACT

A therapeutic method utilizing an implantable neurostimulator device, in which low frequency wave forms, are applied by a configured pulse generator therein through multiple electrodes to targeted networks of interacting brain regions suffering from abnormal communication connections, particularly the central executive network, the default node network, and/or the salience network, to reinstate a normal correlated or an anticorrelated communication connection within and between such networks, and thereby treat brain-related disorders.

### CROSS-REFERENCE TO RELATED APPLICATIONS

This Nonprovisional Application is based on, claims priority to, and incorporates by reference in its entirety for all purposes, U.S. Provisional Application Ser. No. 63/464,182, filed May 4, 2023, which is entitled ‘TRIPLE NETWORK MODULATION’.

### FIELD OF THE INVENTION

The present invention relates to an implantable neurostimulation method that can be utilized to treat central nervous system neurological, psychological, and/or psychiatric conditions, states, and/or disorders mediated by the central nervous system. More particularly, and not by way of limitation, the present invention is directed to a method for using one or more stimulation signals or waves in order to normalize activity and connectivity within and between disrupted brain networks, to treat neurological and/or psychological, and/or psychiatric conditions and/or disorders.

### BACKGROUND OF THE INVENTION

Adverse conditions may be associated with various functions of a brain structure, for example the cortex, or the thalamus. Such conditions may have been treated effectively by delivering electrical energy to one or more target areas of the brain. One method of delivering electrical energy to the brain involves inserting an electrical stimulation lead through a burr hole formed in the skull and then positioning the lead in a precise location proximate a target area of the brain to be stimulated such that stimulation of the target area causes a desired clinical effect. For example, one desired clinical effect may be cessation of tremor from a movement disorder such as Parkinson's Disease. A variety of other clinical conditions may also be treated with deep brain stimulation, such as essential tremor, tremor from multiple sclerosis or brain injury, or dystonia or other movement disorders. The electrical stimulation lead implanted in the brain is connected to an electrical signal generator implanted at a separate site in the body, such as in the upper chest. Current prior art techniques in brain stimulation are still largely based on a phrenological approach that a single brain target can treat a brain disorder. Even though a single target can modulate an entire network, research in network science reveals that many brain disorders are the consequence of maladaptive interactions between multiple networks rather than a single network. Consequently, targeting the main connector hubs of those multiple interacting networks involved in a brain disorder is theoretically more beneficial. We have thus conceived of a next-generation network of neuromodulatory implts implants, that will rely on distributed, multisite neuromodulation to effect change in the brain, offering more precise and localized stimulation to targeted interacting brain networks.

Implantable stimulation systems are devices that generate electrical pulses and deliver the pulses to nervous tissue to treat a variety of disorders. Implantable stimulation can also be performed with magnetic, sound or light stimuli. IMDs are capable of being used all over the nervous system, both at the brain's cortex, the deeper brain structures, hence called DBS or deep brain stimulation, and at the level of the brainstem, spinal cord, dorsal root ganglion or peripheral nerves, either both somatic and autonomic. Basically, every part of the nervous system has been targeted by electrical stimulation and more recently also by magnetic, ultrasound or optic stimulation. Electrical stimulation of the nervous system not only modulates activity of neurons or nerve cells, but also glial cells, such as astrocytes, microglial cells, oligodendroglial cells or Schwann cells.

Recently, new stimulation configurations such as burst stimulation and high frequency stimulation, have been developed, in which closely spaced high frequency pulses are delivered. In general, conventional neurostimulation systems seek to manage pain and other pathologic or physiologic disorders through stimulation of select nerve fibers that carry pain related signals. However, nerve fibers and brain tissue carry other types of signals, not simply pain related signals. Although some central nervous system disorders have been treated through known neurostimulation methods, many other central nervous system disorders exhibit physiological complexity, functional complexity, or other complexity and have not been adequately treated through known neurostimulation methods.

### SUMMARY OF THE INVENTION

In summary, the most preferred embodiment of the invention as claimed comprises a therapeutic method of treating a brain-related disorder in a patient in need of such treatment, comprising the steps of determining the presence of an abnormal correlation interaction or an abnormal anticorrelation interaction between at least one or more nodes of a first identified network of interacting brain regions of interest in the patient with at least one or more nodes of one or more other identified networks of interacting brain regions of interest, and determining whether to reinstate a desired normal correlated or anticorrelated interaction therefore in the patient or a desired normal correlation and/or anticorrelation interaction therefore in the patient. A practitioner may set first parameters that define a carrier waveform, wherein the carrier waveform exhibits an infraslow (0.0-0.1 Hz) or slow (0.1-1 Hz) selected waveform frequency. Then, the practitioner may set second parameters that define a high frequency waveform, that is nested upon the carrier waveform, and wherein at least one each of the carrier waveforms and the high frequency waveform are defined to correspond to physiologic neural oscillations that are known in the field to be associated with at least one of the identified networks of the interacting brain regions of interest. The practitioner may then provide, preferably surgically, one or more pulse generators that are configured to generate a plurality of nested stimulation electrical waveforms that are defined so as to promote or reinstate one or more desired normal correlation or correlated interactions and/or normal anticorrelation or anticorrelated interactions in the patient. Promotion or reinstatement of normal anticorrelated or anticorrelation and/or normal correlated or correlation interactions may be achieved in the claimed method of the invention by delivering a first nested stimulation waveform through one or more electrodes of the pulse generator or generators to the first identified network of interacting brain regions of interest, and then delivering a second nested stimulation waveform through one or more electrodes of the pulse generator or generators to the one or more other identified networks of interacting brain regions of interest in the patient.

This method may be applied without the nested waveforms, i.e. by turning the power of the nested waveforms to zero. Consequently, only the infraslow and slow anticorrelated or anticorrelation or correlated or correlation waveforms are delivered to the brain.

This method can advantageously be extended to comprise the additional step of delivering at least a third or more nested stimulation waveforms through one or more electrodes of the pulse generators to a third or more other identified networks of interacting brain regions of interest in the patient.

In an alternative embodiment a method of treating a neurological, psychological or psychiatric disorder in a patient in need thereof may comprise identifying abnormal communication connectivity between at least two or more identified networks of interacting brain regions of interest in the patient, where the communication connectivity has been disrupted from a normal level of communication activity and that disruption is known in the field to be related to a certain disorder, then determining whether the disrupted communication connectivities are an abnormal communication connectivity correlation or correlated interaction or an abnormal communication connectivity anticorrelation or anticorrelated interaction between a first identified network of interacting brain regions of interest in the patient with one or more other identified networks of interacting brain regions of interest. The practitioner may then set first parameters that define a carrier waveform, wherein the carrier waveform exhibits an infraslow (0.01-0.1 Hz) or slow (0.1-1 Hz) selected waveform, and then sets second parameters that define a high frequency waveform, which is nested upon the carrier waveform, and wherein at least one of the carrier waveform and the high frequency waveform are defined to correspond to physiologic (tonic, burst, noise) neural firing rates, local field potentials or oscillations associated with at least one of the networks of interacting brain regions of interest, then provides or obtains and operates at least one pulse generator which is configured so as to generate a plurality of the nested stimulation electrical waveforms, which individually are comprised of the carrier waveform and of the high frequency waveform, and are configured so as to reinstate one or more desired normal communication connectivity correlation interactions or normal communication connectivity anticorrelation interactions in the patient. Having set the pulse generator(s) configurations, the practitioner may then reinstate normal anticorrelated or normal correlated communication connectivity interactions by delivering to the patient a first nested stimulation waveform through one or more applied electrodes of the pulse generator to a first network of interacting brain regions of interest and by delivering a second nested stimulation waveform through one or more applied electrodes of the pulse generator to another identified network of interacting brain regions of interest.

There may be medical conditions in a patient where the patient is in need of being treated with a third or more additional other nested stimulation waveforms, and for such a patient the practitioner delivers a third or more nested stimulation waveforms through one or more applied electrodes of the pulse generator to a third or more additional other networks of interacting brain regions of interest in the patient. Other networks include, but are not limited to the ventral attention network, dorsal attention network, memory network, emotion/affective network, mirror neuron network, auditory network, visual network, somatosensory network, vestibular network, motor network, or central autonomic nervous system network.

Where a superimposed high frequency signal is used together with an infraslow or slow waveform, one or more of the nested stimulation electrical waveforms may be pink noise, brown noise, red noise, black noise, grey noise, white noise, blue noise, violet noise, or green noise. More particularly, the nested stimulation electrical waveforms may be quantitatively defined by the relationship 1/f^{α}, with f being wave frequency and α being any number between −5 and +5, or is defined as a composite of electrical waveforms of 1/f^{α} and 1/f^{-α}, with f being wave frequency and a being any number between −5 and +5. The infraslow or slow waveform may have any suitable shape, including e.g. a squared or a sinusoidal shape.

The nested noise stimulation electrical waveforms may be generated in a pseudo-random manner. In some embodiments of the invention, the nested noise stimulation waveforms may comprise pink noise nested on a carrier wave form. Alternatively, the nested noise stimulation waveforms may comprise grey noise nested on a carrier wave form.

The networks of interacting brain regions may be selected from the central executive network, the default node network, or the salience network. These networks may be further specifically target by said networks as the left and/or right central executive network, the left and/or right default node network, or the left and/or right salience network. Additionally, networks may be targeted in a strategy wherein the first identified network is targeted as though it is comprised of both the central network and the salience network, and the other identified network is comprised of the default mode network.

The method of the invention may target abnormal connectivity that communicates between the default mode network and the central executive network. Abnormal connectivity between at least the three networks of interacting brain regions described here is believed to be characteristic of the brain related disorders that can include disease attention deficit hyperactivity disorder, anxiety, depression, bipolar disorder, autism, obsessive compulsive disorder, post-traumatic stress disorder syndrome, or schizophrenia, as well as mild cognitive impairment, dementias (Alzheimer, Lewy-body disease, multi-infarct) but also in thalamocortical dysrhythmias (tinnitus, pain, Parkinson Disease), epilepsy, and disorders of consciousness (minimally cognitive state, vegetative state/unresponsive wakefulness syndrome, epilepsy, immune disorders meidasted through the innervation of the spleen and bone, autonomic dysfunctions leading to gastroinstesinal, urogenigal, and cardiorespiratory problems, stress.

Favorable outcomes may be achieved when an abnormal connectivity communication is restored to a normal anticorrelated communication between the central executive network and the salience network, or between the default mode network and the central executive network, or between the salience network and the default mode network.

Favorable outcomes may be achieved when an abnormal connectivity communication is restored to a normal correlated communication between the central executive network and the salience network, or between the default mode network and the central executive network, or between the salience network and the default mode network, or between any other suitable brain networks. Embodiments of the invention may contemporaneously deliver first, second, and third stimulation waveforms to a patient.

Furthermore, the phases of the wave forms, or phase differences between waveforms, can be configured to reinstate an abnormal connectivity communication to a normal anticorrelated communication by configuring an infraslow or slow carrier wave form to be opposite in phase between the default mode network and central executive network, or between the default mode network and the salience network, or between the central executive network and the salience network. Conversely, the phases of the wave forms, or phase differences between waveforms, can be configured to reinstate an abnormal connectivity communication to a normal correlated communication by configuring an infraslow or slow carrier wave form to be opposite in phase between the default mode network and central executive network, or between the default mode network and the salience network, or between the central executive network and the salience network.

The phases of the wave forms can conversely be configured to reinstate an abnormal connectivity communication to a normal anticorrelated communication by configuring an infraslow or slow carrier wave form to be in phase between the default mode network and the central executive network, or between the default mode network and the salience network, or between the central executive network and the salience network. Conversely, the phases of the wave forms can be configured to reinstate an abnormal connectivity communication to a normal correlated communication by configuring an infraslow or slow carrier wave form to be in phase between the default mode network and the central executive network, or between the default mode network and the salience network, or between the central executive network and the salience network.

In one embodiment of the invention as claimed, the patient may be treated with grey noise stimulation to achieve a normalized anticorrelation connectivity communication between the left central executive network, the left default mode network, and the right salience network. in another embodiment this may be bilaterally for all networks or any combination of left and right networks. This may apply to normalizing abnormal correlated, uncorrelated, or anticorrelated activity within a single network or normalizing abnormal correlated, uncorrelated, or anticorrelated activity between 2 or more different networks.

A central nervous disorder, as this term is being used herein, encompasses neurological and psychiatric disorders but also disorders that are controlled or mediated by the central nervous system, such as immune disorders and hormonal disorders in which the central nervous system control of the immune or hormonal system has failed or is failing. In accordance with the methods and apparatus of the present invention, following an initial clinical exam and initial diagnosis, a practitioner must then set first parameters that define a carrier waveform, wherein the carrier waveform exhibits an infraslow or slow selected waveform frequency. Then, the practitioner sets second parameters that define a high frequency waveform, that is nested upon the carrier waveform, and wherein at least one each of the carrier waveform and the high frequency waveform are defined to correspond to physiologic neural oscillations that are known in the field to be associated with at least one of the identified networks of the interacting brain regions of interest. The practitioner then provides one or more implantable pulse generators that are configured to generate a plurality of nested i.e. low carrier waveform and high information stimulation electrical sound/ultrasound, optic or magnetic waveforms that are defined so as to reinstate one or more desired normal correlation interactions or normal anticorrelation interactions in the patient between 2 or more networks. These networks are most preferably the 3 cognitive networks, i.e. the central executive network, the default mode network and the salience network, but are not limited to the cognitive networks. The networks can involve any other 2 or more networks and can extend to 4 or 5 networks, including the emotional/affective network, somatosensory, medial, lateral and descending pain, auditory, vestibular, visual, olfactory, gustatory, social networks as well as the mirror neuron network, dorsal attention network, ventral attention network, reward and dysreward network, the central sympathetic and parasympathetic network, or any network denoting these networks but given a different name. For example, the mentalizing network overlaps largely or is similar to the default mode network. Under the theory of mind this network includes the default mode network with added VLPFC. The social network involves the theory of mind network with added STS(=social cortex). Reinstatement of normal anticorrelated or normal correlated interactions is achieved in the claimed method of the invention by delivering a first infraslow or slow stimulation waveform through one or more electrodes of the pulse generator or generators to the different identified interacting brain regions of interest, and then delivering a second nested higher frequency stimulation waveform through one or more electrodes of the pulse generator or generators to the one or more other identified networks of interacting brain regions of interest in the patient, in which the infraslow or slow component is in phase, i.e. correlated, or in antiphase, i.e. anticorrelated, or uncorrelated.

In another embodiment the infraslow/slow component may be presented without a specific phase, which results in a disruption of the abnormal correlated or anticorrelated communication. Some examples of the high frequency waveforms that can be nested on the infraslow or slow carrier waves include, but are not limited to, different noise waveforms, burst waveforms, and tonic waveforms. Most commonly and more preferably, the infraslow/slow and faster waveforms will be of delivered through the same electrodes, through this is not the exclusive approach to delivery. The difference with traditional nested stimulation (see DeRidder, U.S. Pat. No. 7,734,340, the entire disclosure of which is incorporated herein by reference) is that in the present invention the nesting occurs on infraslow or slow waves that are either in phase or in antiphase or out of phase and are normalizing or disrupting the infraslow/slow phase relationships, which is a point of unobvious novelty of this invention. This method can advantageously be extended to comprise the additional step of delivering at least a third or more nested stimulation waveforms through one or more electrodes of the pulse generators to a third or more other identified networks of interacting brain regions of interest in the patient. One non-limiting example is to add burst waveforms to the noise waveforms that are already used as nested waveforms on the correlated or anticorrelated infraslow and/or slow waveforms.

In a first embodiment of the claimed invention, it can be practiced as a method of treating a central nervous system disorder in a patient in need thereof, comprising the steps of identifying abnormal communication connectivity between the nodes or brain areas that make up one known network of interacting brain regions of interest in the patient, where the communication connectivity has been disrupted from a normal level of communication activity and that disruption is known in the field to be related to a certain central nervous system disorder, then determining whether the disrupted communication connectivities are an abnormal communication connectivity correlation interaction or an abnormal communication connectivity anticorrelation interaction between the interacting brain regions of the network of interest. An example is the determination of whether all the brain regions that normally make up the default mode are still correlated in activity, and if not, then applying the method of the present invention. This method could apply to other networks such as the emotional/affective network, somatosensory, medial, lateral and descending pain, auditory, vestibular, visual, olfactory, gustatory, social networks as well as the mirror neuron network, dorsal attention network, ventral attention network, reward and dysreward network, the central sympathetic and parasympathetic network, or any network denoting these networks but given a different name. For example, the mentalizing network overlaps largely or is similar to the default mode network. The theory of mind network includes the default mode network with added VLPFC. The social network involves the theory of mind network with added STS(=social cortex).

In an alternative preferred embodiment of the claimed invention, it can be practiced as a method of treating a central nervous system disorder in a patient in need thereof, comprising the steps of identifying abnormal communication connectivity between at least one, or more, identified regions of interacting brain networks of interest in the patient, where the communication connectivity has been disrupted from a normal level of communication activity and that disruption is known in the field to be related to a certain central nervous system disorder, then determining whether the disrupted communication connectivities are an abnormal communication connectivity correlation interaction or an abnormal communication connectivity anticorrelation interaction between a first identified network of interacting brain regions of interest in the patient with one or more other identified networks of interacting brain regions of interest. The practitioner then sets first parameters that define a carrier waveform, wherein the carrier waveform exhibits an infraslow or slow selected waveform frequency in a range of frequencies of up to 1 Hz, and then setting second parameters that define a high frequency waveform, which is nested upon the carrier waveform, and wherein at least one of the carrier waveform and the high frequency waveform are defined to correspond to physiologic neural oscillations or firing modes associated with at least one of the regions of interacting brain networks of interest, then provides or obtains and operates at least one pulse generator which is configured so as to generate a plurality of the nested stimulation electrical waveforms, which individually are comprised of the carrier waveform and of the high frequency waveform, and are configured so as to reinstate one or more desired normal communication connectivity correlation interactions or normal communication connectivity anticorrelation interactions in the patient. Having set the pulse generator(s) configurations, the practitioner may then reinstate normal anticorrelated or normal correlated communication connectivity interactions by delivering to the patient a first nested stimulation waveform through one or more applied electrodes of the pulse generator to a first region of interacting brain neetworks of interest and by delivering a second nested stimulation waveform through one or more applied electrodes of the pulse generator to an other identified network of interacting brain regions of interest. In another alternative preferred embodiment, the phases of the infraslow and/or slow carrier waveforms may be randomly activated as to break abnormal correlated activity that does not require anticorrelation but uncorrelation or no correlation. Thus, the 2 or more networks may become uncorrelated, neither correlated nor anticorrelated.

There may be medical conditions in a patient where the patient is in need of being treated with a third or more additional other nested stimulation waveforms, and for such a patient the practitioner delivers a third or more nested stimulation waveforms through one or more applied electrodes of the pulse generator to a third or more additional other networks of interacting brain regions of interest in the patient.

In another alternative preferred embodiment of the claimed invention, the practitioner engages the method of treating a central nervous system disorder in a patient, comprising the steps of identifying abnormal connectivity between at least two or more identified networks of interacting brain regions, where connectivity has been disrupted from the connectivity's normal communication activity, and the disruption is known in the field as being related to a central nervous system disorder, recording electrical activity related to neural activity in the brain of the patient, and with reference to the recording, determining the presence of an abnormal correlation interaction or the presence of an abnormal anticorrelation interaction between each identified network of interacting brain regions, with each of the remaining other identified networks of interacting brain regions, and determining whether the patient needs to have the reinstatement of a normal anticorrelated or a normal correlated interaction. The practitioner then sets a first set of parameters that define a stimulation carrier waveform, wherein the carrier waveform exhibits an infraslow or slow selected waveform frequency in a range of frequencies of up to 1 Hz, and then sets a second set of parameters that define a stimulation high frequency waveform, where the stimulation high frequency waveform is nested upon the stimulation carrier waveform, and wherein at least one of the stimulation carrier waveform and the stimulation high frequency waveform are defined to correspond to physiologic neural oscillations that are known in the field to be associated with at least one of the networks of the interacting brain regions. Having set the wave form parameters, the practitioner then provides or obtains an implantable stimulator to generate a plurality of nested stimulation electrical waveforms, which individually are comprised of the stimulation carrier waveforms and of the high frequency stimulation waveforms, wherein the nested stimulation waveform comprises a plurality of pulse bursts, and wherein the pulse bursts are characterized in that each of the pulse bursts is comprised of a plurality of discrete pulses, and furthermore that the plurality of discrete pulses within each pulse burst are repeated according to a frequency parameter of the high frequency waveform, and a wave amplitude of each of the discrete pulses, within respective such pulse bursts, is controlled according to the high frequency waveform nested on the carrier waveform such that wave amplitude peaks of the corresponding plurality of said discrete pulses vary within each respective one of the pulse burst(s). As the term is used herein, a ‘burst’ is a waveform in which closely spaced monophasic high frequency pulses are delivered that are charge balanced after the high frequency pulses are delivered. The number of pulses within a burst can be anywhere from 2 to 20 pulses, delivered at a frequency of 20 to 1000 Hz. The frequency of the bursts is between 0.01 Hz to 100 Hz. In some embodiments the nested bursts may be charged balanced for every pulse. The practitioner may then reinstate normal anticorrelated or normal correlated interactions by delivering a first nested stimulation waveform through one or more applied pulse generator electrodes to a first network of interacting brain regions of interest, and delivering a second nested stimulation waveform through one or more applied pulse generator electrodes to a second network of interacting brain regions of interest.

There may be medical conditions in a patient where the patient is in need of being treated with a third or more additional other nested stimulation waveforms, and for such a patient the practitioner delivers a third or more nested stimulation waveforms through one or more applied electrodes of the pulse generator to a third or more additional other networks of interacting brain regions of interest in the patient.

In the alternative embodiments of the invention as claimed, one or more of the nested stimulation electrical waveforms may be pink noise, brown noise, red noise, black noise, grey noise, white noise, blue noise, violet noise, or green noise. More particularly, the nested stimulation electrical waveforms may be quantitatively defined by the relationship 1/f^{α}, with f being wave frequency and a being any number between −5 and +5, or is defined as a composite of electrical waveforms of 1/f^{α} and 1/f^{-α}, with f being wave frequency and a being any number between −5 and +5.

The nested noise stimulation electrical waveforms may be generated in a pseudo-random manner. In a more preferred embodiment of the invention, the nested noise stimulation waveforms comprises pink noise nested on a carrier wave form. Alternatively, the nested noise stimulation waveforms comprises grey noise nested on a carrier wave form.

In another embodiment the nested waveforms may also be a narrow band noise of the known frequency bands, i.e. infraslow (0.01-0.1 Hz), slow (0.1-1 Hz), delta (1-3 Hz), theta (4-7 Hz), alpha (8-12 Hz), beta (13-30 Hz), gamma (30-80 Hz), or high frequency noise (>80 Hz). The high frequency bands have been split up in fast (80-200 Hz) and ultrafast (200-1000 Hz). This includes sub bands such as low alpha (=alpha 1) (8-10 Hz) or high alpha (=alpha 2) (10-12 Hz), low beta (=beta 1)(12-16 Hz) beta (=beta 2)(16-20 Hz), and high beta (=beta3) (20-30 Hz). Or the narrow band noise may be mu rhythm (7.5-12.5 Hz) or SMR (12.5-16.5 Hz). The nested narrow band noise may be selected to be any combination of frequencies or frequency bands, e.g. beta plus gamma, delta plus theta, or any other combination. The nested narrow band noises may all have the same power (white noise) or structured following a power law, i.e. 1/fα and 1/f−α, with f being wave frequency and a being any number between −5 and +5.

The networks of interacting brain regions are most preferably selected from the central executive network, the default node network, or the salience network. These networks may be further specifically targeted by said networks as the left and/or right central executive network, the left and/or right default node network, or the left and/or right salience network. Additionally, networks may be targeted in a strategy wherein the first identified network is targeted as though it is comprised of both the central network and the salience network, and the other identified network is comprised of the default mode network. The central executive network is also known as the frontoparietal control network, the salience network as the cingulo-opercular network.

Preferably, the method of the invention targets abnormal connectivity that communicates between the default mode network and the central executive network. Abnormal connectivity between at least the three networks of interacting brain regions described here is characteristic of disease attention deficit hyperactivity disorder, anxiety, depression, bipolar disorder, autism, obsessive compulsive disorder, post-traumatic stress disorder syndrome, or schizophrenia, but the invention is not limited to these disorders, but additionally extends to any disorder associated with abnormal interactions between the said three networks.

Favorable outcomes are achieved when an abnormal connectivity communication is restored to a normal anticorrelated communication between the central executive network and the salience network, or between the default mode network and the central executive network, or between the salience network and the default mode network.

Favorable outcomes can be achieved when an abnormal connectivity communication is restored to a normal correlated communication between the central executive network and the salience network, or between the default mode network and the central executive network, or between the salience network and the default mode network. The preferred embodiments of the invention may contemporaneously deliver first, second, and third nested stimulation waveforms to a patient.

Furthermore, the phases of the infraslow and slow carrier wave forms can be configured to reinstate an abnormal connectivity communication to a normal anticorrelated communication by configuring an infraslow or slow carrier wave form to be opposite in phase between the default mode network and central executive network, or between the default mode network and the salience network, or between the default mode and the central executive network plus the salience network, the altter two being correlated. Conversely, the phases of the wave forms can be configured to reinstate an abnormal connectivity communication to a normal correlated communication by configuring an infraslow or slow carrier wave form to be opposite in phase between the default mode network and central executive network, or between the default mode network and the salience network, or between the default mode and the central executive network plus the salience network, the latter two being correlated. These phases can be configured to be randomly generated in each of the three networks, as to create a disruption between these three networks, followed by a second phase of resetting a phase as described above, i.e. anticorrelation between default mode network and salience network plus central executive network. In another alternative embodiment the salience network and default mode network may be programmed such that they are phase correlated in the slow or infraslow carrier waves as to induce increase in internally oriented thought.

The phases of the wave forms can conversely be configured to reinstate an abnormal connectivity communication to a normal anticorrelated communication by configuring an infraslow or slow carrier wave form to be in phase between the default mode network and central executive network, or between the default mode network and the salience network, or between the central executive network and the salience network. The configuration thus embodies a correlation between the default mode, salience network and central executive network. Conversely, the phases of the wave forms can be configured to reinstate an abnormal connectivity communication to a normal correlated communication by configuring an infraslow or slow carrier wave form to be in phase between the default mode network and central executive network, or between the default mode network and the salience network, or between the central executive network and the salience network.

In a preferred embodiment of the invention as claimed, the patient is treated with grey noise stimulation as nested stimulation to achieve a normalized anticorrelation connectivity communication between the left central executive network, the left default mode network, and the right salience network.

### BRIEF DESCRIPTION OF THE FIGURES

FIG. 1A is an illustration of altered connectivity involving the central executive network.

FIG. 1B illustrates altered connectivity involving the default mode network.

FIG. 2 illustrates hypoconnectivity and hyperconnectivity between brain networks.

FIG. 3 shows a stimulation wave according to one embodiment, including an infraslow or slow carrier waveform with a nested or superimposed noise waveform. This figure also illustrates various colors, or types of noise waveforms.

FIG. 4 illustrates correlation and anticorrelation between brain networks.

FIG. 5 shows how stimulation waves may be applied to brain networks to ameliorate abnormal correlation or anticorrelation conditions. Normalization of triple network interactions via infraslow stimulation may be combined with pink noise nested on top of an infraslow stimulus. The infraslow component permits correlation activity within networks and between the salience and central executive network. It also permits anticorrelation activity between the default mode and the salience and central executive network by supplying infraslow stimuli in antiphase at the default mode and the salience plus central executive network. The pink noise nested on top of this infraslow component serves to mimic normal physiological brain activity.

FIG. 6 illustrates expected outcomes with a delayed start study protocol.

FIG. 7 shows the results from a study based on the Montgomery-Asberg Depression Rating Scale (MADRS), showing the improvement of depression symptoms with treatment over placebo. On this scale, 0-6 is considered normal, no symptoms; 7-19 is considered mild depression; 20-34 is considered moderate depression; and over 34 is considered severe depression.

FIG. 8 shows the results from a study based on a Quick Inventory of Depressive Symptomatology (QIDS) self-report. On this scale, ≤5 represents no depression; 6-10 is considered mild depression; 11-15 is considered moderate depression; 16-20 is considered severe depression and ≥21 is considered very severe depression.

FIG. 9 shows the results from a study based on the Ruminative Response Scale (RRS).

FIG. 10 shows a questionnaire used in the WHO-5 Well Being Index.

FIG. 11 shows the results of a study based on the WHO-5 Well Being Index.

FIGS. 12 and 13 show the results of a study based on the Hamilton Anxiety and Depression Scale (HADS) On this scale, 0-7 represents no anxiety, no depression; 8-10 indicates mild symptoms; 11-14 indicates moderate symptoms; and 15-21 indicates severe symptoms.

FIGS. 14 and 15 show the effects of the level of noise on functional connectivity. Stochastic resonance effect of noise stimulation can both increase and decrease connectivity. With low amplitudes no change in connectivity ensues as the firing threshold is not reached (weak noise in FIG. 14). Optimal amplitudes push activity at different areas above threshold, leading to increased connectivity (optimal noise FIG. 14). High amplitudes create noise at the two areas, preventing phase synchronization and thus functional connectivity (high noise FIG. 14). This creates an inverted U curve profile in connectivity (FIG. 15).

FIG. 16 shows a method acting on four networks, such as might be used in treating, for example, Anxiety, depression, PTSD or schizophrenia.

FIG. 17 illustrates a method acting on three networks plus two examples of a fourth network, for treating pain or tinnitus.

FIG. 18 illustrates one example of a neuromodulation system schematic diagram.

FIG. 19 is a depiction illustrating how the different implantable devices of the invention are connected to the clinician.

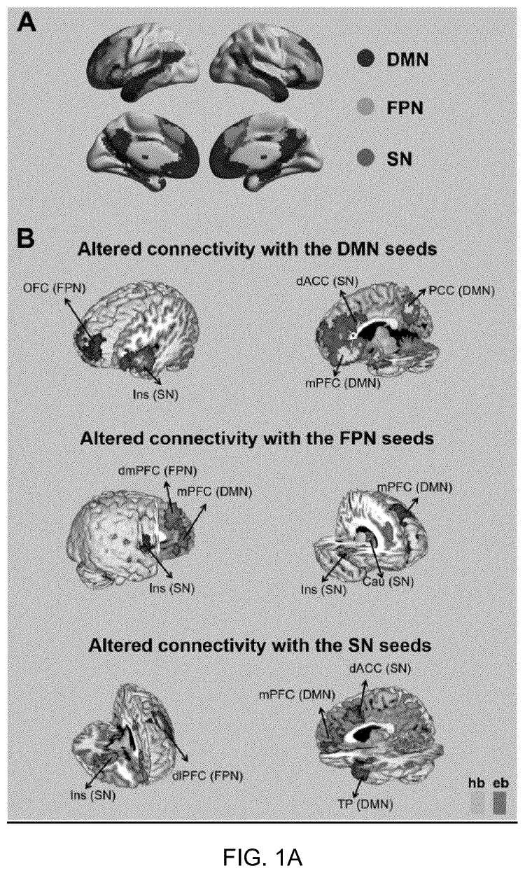

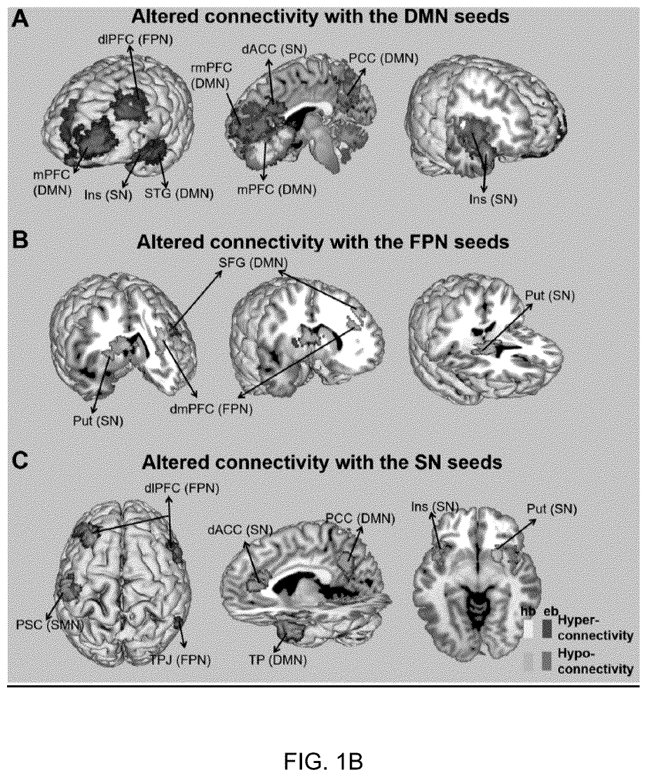

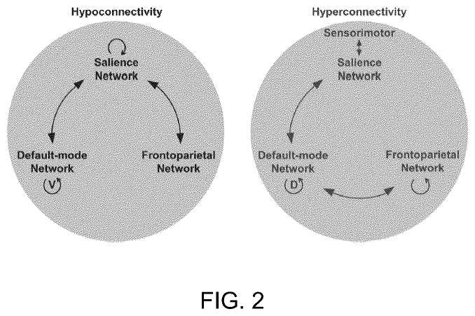

### DETAILED DESCRIPTION OF THE INVENTION AND OF THE FIGURES

While multiple embodiments are described, still other embodiments of the described subject matter will become apparent to those skilled in the art from learning from the following detailed novel disclosure descriptions and drawings, which show and describe illustrative embodiments of disclosed inventive subject matter as claimed. As will be realized, the inventive subject matter is capable of modifications in various aspects, all without departing from the spirit and scope of the described subject matter. Accordingly, the drawings and detailed description are to be regarded as illustrative in nature and not restrictive.

Definitions. Wherever used herein, the usage of the word “a” or “an”, when used in conjunction with the term “comprising” in the claims and/or the specification, shall mean “one,” or “one or more,” or “at least one,” or “one or more than one.” The terms “having”, “including”, “containing”, and “comprising” are interchangeable and one of skill in the art may readily recognize that these terms are open-ended terms. If a term is not defined in this specification, then technical and scientific terms used herein have the same meaning as commonly understood by one of ordinary skill in the art to which this invention belongs. For purposes of the descriptions of the invention, the following terms are defined below.

Action Potential. An action potential is a depolarization that occurs when the membrane potential of a specific cell location rapidly rises and falls; membrane potential here means the difference in electric potential between the interior and the exterior of a biological cell. The terms “pulse” and “spike” are used interchangeably herein to refer to an action potential.

Acute pain. Acute pain refers to a relatively recent onset of pain, pain associated with an injury or trauma, or immediate pain triggered by injury. Acute pain can also be referred to as “phasic.” Generally, acute pain is associated with a greater intensity of pain and/or one or more impairments in functionality for the person.

Affective disorders. Affective disorders will refer to a group of disorders that are commonly associated with co-morbidity of depression and anxiety symptoms.

Alpha. Alpha refers to a brainwave neural oscillation falling into a frequency band in the range of from about 8 to about 12 Hz.

Anticorrelated. The term anticorrelated narrowly means a 180 degree phase lag, but in the present disclosure is to betaken more broadly as constituting a range of from 90 degrees to 270 degrees.

Beta. Beta refers to a brainwave neural oscillation falling into a frequency band in the range of from about 1.3 to about 30 Hz.

Brain networks. Wherever a brain network is named as an element of the method or devices of the invention, the network shall be understood and interpreted to be describing and claiming each region, subregion, system or subsystem of such network, both in the anatomical sense thereof, and/or in the functional sense thereof, all as known to those of ordinary skill in the art. All brain regions in a network show correlated activity, behaving as one structural or functional unity. Brain networks further specifically include the central executive network, the default node network, the salience network, the emotional network comprised of the amygdala, the subgenual anterior cingulate cortex, and the orbitofrontal cortex, the ventral attentional network comprised of the inferior parietal, and ventrolateral prefrontal networks, and the dorsal attentional network comprised of the premotor, and the superior parietal networks, The Mirror neuron network comprised of the ventrolateral prefrontal, inferior parietal, and the superior temporal sulcus, the sensorimotor networks comprised of the visual, auditory, somatosensory, vestibular, and motor networks, the speech network comprised of the Wernicke and Broca areas, the central autonomic (i.e. control) network comprised of the insula, amygdala, anterior cingulate, posterior cingulate/precuneus networks, the brainstem, the hypothalamus, the mediodorsal thalamus, the periaqueductal grey, and the ventral tegmental areas. When triple networks are discussed this will preferably mean the central executive network, the default node network, and the salience network. A triple network treatment methodology may be extended to the inclusion of a fourth network and may be extended to the inclusion of a fifth network.

Brain-related disorder. A brain-related disorder as that term is used herein is any illness, disease or disorder than can be linked to abnormal functioning of the brain. This encompasses mental, psychological or psychiatric disorders, but also neurological and autonomic nervous system disorders, as well as immune disorders, and endocrinological disorders.

Burst. The term burst refers to a period in a spike train that has a much higher discharge rate than surrounding periods in the spike train (N. Urbain et. al., 2002) Thus, burst can refer to a plurality of groups of spike pulses. A burst is a train of action potentials that, possibly, occurs during a ‘plateau’ or ‘active phase’, followed by a period of relative quiescence called the ‘silent phase’ (Nunemaker, Cellscience Reviews Vol 2 No. 1, 2005.) Thus, a burst comprises spikes having an inter-spike interval in which the spikes are separated by about or approximately 0.5 milliseconds, or a time period that is functionally equivalent, to about or approximately 100 milliseconds, or a time period that is functionally equivalent. Those of skill in the art realize that the inter-spike interval can be longer or shorter. Yet further, those of skill in the art also realize that the spike rate within the burst does not necessarily occur at a fixed rate, this rate can be variable. A burst in general is considered to be a waveform in which closely spaced monophasic high frequency pulses are delivered that are charge balanced after the high frequency pulses are delivered. The number of pulses within a burst can be anywhere from 2 to 20 pulses, delivered at a pulse or spike frequency of 20 to 1000 Hz. The frequency of the bursts is between 0.01 Hz to 100 Hz. In some embodiments the nested bursts may be charged balanced for every pulse.

Burst Firing or Burst Mode. Burst mode refers to an action potential that is a burst of high frequency spikes (e.g. in the range of about 400-1000 Hz) (Beurrier et al., 1999). Burst firing acts in a nonlinear fashion with a summation effect of each spike. One skilled in the art is also aware that burst firing can also be referred to as phasic firing, rhythmic firing (Lee 2001), pulse train firing, oscillatory firing and spike train firing, and all of these terms as may be used herein are interchangeable.

Burst Spike, A burst spike refers to a spike that is preceded or followed by another spike within a short time interval (Matveev, 2000), in other words, at a high frequency, between 50 and 1000 Hz. this is associated with a short inter-spike interval, in which this interval is generally about 100 ms but can be shorter or longer, for example 0.5 milliseconds.

Central Executive Network (CEN). The central executive network (CEN), generally also known among those of ordinary skill in the art as the frontoparietal control network, the frontoparietal network (FPN) or, more specifically, the lateral frontoparietal network (L-FPN), is a large-scale brain network primarily composed of the dorsolateral prefrontal cortex and the posterior parietal cortex around the intraparietal sulcus as well as the temporo-occipital junction. Itis involved in cognitive functioning, complex problem-solving and working memory, or in other words, in goal-oriented behavior. The CEN is one of three networks referred to in the field as the so-called triple-network model, along with the salience network (SN) and the default mode network (DMN). The salience network facilitates switching between the CEN and DMN. The CEN is primarily composed of the rostral lateral and dorsolateral prefrontal cortex (especially the middle frontal gyrus) and the anterior inferior parietal lobule, as well as the occipitotemporal junction. Additional regions include the middle cingulate gyrus and potentially the dorsal precuneus, posterior inferior temporal lobe, dorsomedial thalamus and the head of the caudate nucleus.

Central Nervous System. The central nervous system (CNS) comprises the brain and spinal cord, which together function as the principal integrator of sensory input and motor output. In general terms, the brain consists of the cerebrum (cerebral hemispheres and the diencephalons), the brainstem (midbrain, pons, and medulla); and the cerebellum. It is well known that the cerebrum represents the highest center for sensory and motor and emotional and cognitive processing. In general, the frontal lobe processes motor, visual, speech, and personality modalities; the parietal lobe processes sensory information; the temporal lobe, auditory and memory modalities; and the occipital lobe vision. The cerebellum, in general, coordinates smooth motor activities and processes muscle position, while the brainstem conveys motor and sensory information and mediates important autonomic functions. These structures are of course integrated with the spinal cord which receives sensory input from the body and conveys somatic and autonomic motor information to peripheral targets. Thus, one of skill in the art realizes that the central nervous system is capable of evaluating incoming information and formulating response to changes that threaten the homeostasis of the individual.

Central Nervous System related disorder. The term Central Nervous System Disorders will include any disorder related to abnormal functioning of the central nervous system, including psychological, psychiatric, or neurological disorders, and furthermore including immunological and endocrine or hormonal disorders insofar as the central nervous system control of the immunological and endocrine system is dysfunctional. Thus, central nervous system related disorders also include psychoneuroimmunological disorders as well as psychoneuroendocrine disorders, and somatoformn disorders.

Central neuronal tissue. Central neuronal tissue refers to neuronal tissue associated with the brain, spinal cord or brainstem.

Chronic Pain. As used herein, the term chronic pain can generally be characterized as being nociceptive or non-nociceptive including neuropathic pain. Yet further, it can also be characterized as pain that has lasted for a sustained period of time, for example, more than three months. Chronic pain generally also has significant psychological and emotional affects and with the passage of time can limit a person's ability to fully function.

Deafferentation. As used herein, the term deafferentation refers to a loss of the sensory input normally generated from a portion of the body.

Delta. The Delta brainwave frequency band falls into the range of from about 1 Hz to about 4 Hz.

Determining or identifying correlation or anticorrelation. As used herein, this can be determined or identified by the process of recording brain activity using EEG, MEG, fMRI, NIRS or any other method of recording brain activity and connectivity. However, it can also be determined theoretically, to the extent that published research has established that abnormal correlations and/or anticorrelations exist between areas within a network or between multiple networks in specific central nervous system related disorders.

Dementia. As used herein, the term dementia refers to the loss of cognitive and intellectual functions without impairment of perception or consciousness. Dementia is typically characterized by disorientation, impaired memory, judgment, and intellect, and by a shallow labile affect. The types of disease that present themselves clinically as a dementia include vascular dementia, frontotemporal dementia, Lewy Body dementia, normal pressure hydrocephalus, young onset dementia, posterior cortical atrophy, corticobasal degeneration, Korsakoff syndrome, mild cognitive impairment, Huntingdon disease, HIV-associated neurocognitive disorder, alcohol-related dementia, progressive supranuclear palsy, or one or more of the secondary dementias. To the extent that any of these clinical presentations are in whole or in part caused or exacerbated by abnormal brain center connectivities or communications, then they are included within the scope of the invention as claimed.

Disorder. Disorder means a functional abnormality or disturbance, and furthermore the term may be used interchangeably with illness, disease, or pathology.

Electroencephalograph. An electroencephalograph, or EEGm is a measurement, detected and displayed by electronic devices well known to those of ordinary skill in the art, of electrical impulses that are generated by collective brain neurons. Such impulses are identifiable as brainwaves falling into different ranges of frequencies. Electrodes connected to the device are placed on specific sites on the scalp to detect and record the electrical impulses that collections of neurons are generating within the brain.

Frequency. Frequency means the number of times a wave repeats itself within a second.

Frontoparietal (control) network. The frontoparietal network (FPN), generally also known among those of ordinary skill in the art as the central executive network (CEN) or, more specifically, the lateral frontoparietal network (L-FPN), is a large-scale brain network primarily composed of the dorsolateral prefrontal cortex and posterior parietal cortex around the intraparietal sulcus. It is involved in sustained attention, complex problem-solving and working memory. The FPN is one of three networks referred to in the field as the so-called triple-network model, along with the salience network (SN) and the default mode network (DMN). The salience network facilitates switching between the FPN and DMN. The FPN is primarily composed of the rostral lateral and dorsolateral prefrontal cortex (especially the middle frontal gyrus) and the anterior inferior parietal lobule. Additional regions include the middle cingulate gyrus and potentially the dorsal precuneus, posterior inferior temporal lobe, dorsomedial thalamus and the head of the caudate nucleus.

Functional Magnetic Resonance Imaging (fMRI). fMRI is a measure of brain activity performed by detecting changes associated with blood flow. This technique relies on the fact that cerebral blood flow and neuronal activation are coupled. When an area of the brain is in use, blood flow to that region also increases.

Gamma. A gamma brainwave frequency band falls into the range of greater than about 30 Hz, and typically, though not exclusively, ranges up to about 100 Hz.

Hz. The Hertz is the unit of measurement of wave frequency, defined as one cycle per second.

Implantable Pulse Stimulation (IPS). By means of an invasive surgical procedural treatment, an implantable pulse generator is inserted that may deliver electrical stimulation to targeted structures in the thalamus, brainstem, or other targeted areas of the brain.

In communication (with). As used herein, the term “in communication” is a phrase that refers to the stimulation lead being adjacent, in the general vicinity, in close proximity, or directly next to or directly on the predetermined stimulation site, for example an area of the cortex, or an area associated with the sensory, cortex, or any subcortical area or structure that is projected onto, into, or out from the sensory cortex, or any identified brain region or area that is determined by mapping the brain of a subject suffering from a neurological condition. Thus, one of skill in the art understands that the lead or electrode is “in communication” with the target tissue or site if the stimulation results in a modulation of neuronal activity resulting in the desired response, such as modulation of the neurological disorder.

Infraslow. An infraslow brainwave frequency band falls into the range of about 0.01 Hz to about 0.1 Hz.

Mammal. The term mammal herein includes, but is not limited to, humans, dogs, cats, horses and cows. However, the terms “mammalian organism,” “subject”, “patient”, or “person” are herein used interchangeably except where the context makes it clear that a human patient or human clinical trial participant is the object of the sentence. Although veterinarian patients are included within the scope of the invention, the most preferred patients are humans.

Medial Frontoparietal Network (M-FPN). The functional network that is the Default Mode Network (DMN) is known anatomically as the Medial Frontoparietal Network.

Modulate. To modulate refers to the ability to positively or negatively regulate neuronal activity. Thus, the term modulate can be used to refer to an increase, decrease, masking, altering, overriding, restoration, phase reversal, correlation alteration, correlation restoration, anticorrelation alteration, or anticorrelation restoration, of neuronal activity.

Neurology or Neurological. Neurology or neurological refer to conditions, disorders, and/or diseases that are associated with the nervous system. The nervous system comprises two components, the central nervous system, which is composed of the brain and the spinal cord, and the peripheral nervous system, which is composed of ganglia and the peripheral nerves that lie outside the brain and the spinal cord. One of skill in the art realizes that the nervous system may be separated anatomically, but functionally they are interconnected and interactive. Yet further, the peripheral nervous system is divided into the autonomic system (parasympathetic and sympathetic), the somatic system and the enteric system. Thus, any condition, disorder and/or disease that effects any component or aspect of the nervous system (either central or peripheral) is referred to as a neurological condition, disorder and/or disease. As used herein, the term “neurological” or “neurology” also encompasses the terms “neuropsychiatric” or “neuropsychiatry” and “neuropsychological” or “neuropsychology”. Thus, for example, a neurological disease, condition, or disorder includes, but is not limited to, affective disorders, Alzheimer's dementia, anxiety, autonomic dysfunctions, bipolar disorder, cognitive disorders, depression, epilepsy, hypertension of neurological origin, mental disorders, migraine headache, movement disorders, pain disorders whether acute or chronic, Parkinson's Disease, prefrontal lobe dementia, schizophrenia, sleep disorders, stroke, or tinnitus, and the like.

Neuromodulation. Neuromodulation is the alteration of nerve activity through targeted delivery of a stimulus, such as electrical, light, magnetic, optical, or sound stimuli, or chemical agent stimulus, to specific central nervous system sites in the body”. It is carried out to improve, modulate, or normalize the function of nervous tissue. Neuromodulation, whether electrical, magnetic, optical, or sound employs the body's natural biological response by stimulating nerve cell activity that can influence populations of nerves by the release of signal molecules such as the neurotransmitters. Examples of neurotransmitters are dopamine, serotonin, or noradrenaline, or other classes of chemical messengers. Neuromodulation can also result in altered release of other chemical messengers such as neuropeptides, which may modulate the excitability and firing patterns of neural circuits. Neuromodulation may also influence electrical synapses such as gap junctions. There may also be more direct electrophysiological effects on neural membranes as the mechanism of action of electrical interaction with neural elements. The end effect is an improvement or a normalization of a neural network function from its perturbed state. Presumed mechanisms of action for neurostimulation include depolarizing blockade, stochastic normalization of neural firing, axonal blockade, reduction of neural firing keratosis, and suppression of neural network oscillations. Further mechanisms include genetic and epigenetic modulation, i.e. the expression of certain genes. Neuromodulation does not only involve changing activity and connectivity of neurons or nerve cells, but also of glial cells such as astrocytes, microglial cells, oligodendroglial cells.

Neuronal and nervous. Neuronal and nervous, are terms that refer to a cell type which is a morphologic and functional unit of the brain, brainstem, spinal cord, and/or peripheral nerves.

Neuropsychiatry or Neuropsychiatric. Neuropsychiatry or neuropsychiatric refers to conditions, disorders and/or diseases that relate to either or both of organic and psychic/mental disorders of the nervous system.

Neuropsychological or Neuropsychologic. Neuropsychological or neuropsychologic or neuropsychology refers to conditions, disorders and/or any disease that relates to the functioning of the brain and the cognitive processes, or processors, or behavior.

Neurostimulation. Neurostimulation is the purposeful modulation of the nervous system's electrical activity using invasive (i.e. surgically implanted microelectrodes) or using non-invasive means. The preferred embodiments of the present invention use invasive surgical means. Neurostimulation shall refer to electromagnetic approaches to neuromodulation, in this invention being through invasive surgical means. The term neurostimulation may be used interchangeably with neuromodulation.

Nociceptive pain. Nociceptive pain involves direct activation of the nociceptors, meaning the mechanical, chemical, and thermal receptors found in various tissues, such as bone, muscle, vessels, viscera, and cutaneous and connective tissue. Nociceptive pain can also be referred to as somatic pain. The afferent somatosensory pathways are thought to be intact in nociceptive pain and examples of such pain include cancer pain from bone or tissue invasion, non-cancer pain secondary to degenerative bone and joint disease or osteoarthritis, and failed back surgery.

Non-nociceptive pain. As used herein, the term “non-nociceptive pain” occurs in the absence of activation of peripheral nociceptors. Non-nociceptive pain can also be referred to as neuropathic pain, or deafferentation pain. Non-nociceptive pain often results from injury or dysfunction of the central or peripheral nervous system. Such damage may occur anywhere along the neuroaxis and includes thalamic injury or syndromes (also referred to as central pain, supraspinal central pain, or post-stroke pain); stroke; traumatic or iatrogenic trigeminal (trigeminal neuropathic) brain or spinal cord injuries; phantom limb or stump pain; postherpetic neuralgia; anesthesia dolorosa; brachial plexus avulsion; complex regional pain syndrome I and II; postcordotomy dysesthesia; and various peripheral neuropathies, inclusive of pain associated with or related to vascular pathology (vasculitis, angina pectoris, etc.) both peripheral vascular pathology, central or cerebral vascular pathology, and/or cardiac vascular abnormalities.

Orbitofrontal cortex (OF). The orbitofrontal cortex (OFC) is a prefrontal cortex region in the frontal lobes of the brain, which is involved in the cognitive process of decision-making. In humans it consists of Brodman areas 10, 11, and 47. The OFC is functionally related to the ventromedial prefrontal cortex. Therefore, the region is distinguished due to the distinct neural connections and the distinct functions it performs. It is defined as the part of the prefrontal cortex that receives projections from the medial dorsal nucleus of the thalamus, and is thought to represent emotion, taste, olfaction and the action of reward in decision making. It gets its name from its position immediately above the orbits in which the eyes are located.

Pain. The term “pain” as used herein refers to an unpleasant sensation or altered sensory perception. For example, the subject experiences discomfort, distress or chronic or acute suffering. Pain of a moderate or high intensity is typically accompanied by anxiety. Thus, one of skill in the art is cognizant that pain may have dual properties, for example sensation and emotion. Examples of pain or altered sensory perception can include, but are not limited to paresthesias, dysesthesias, synesthesla, hyperalgesia, allodynia, phantom perceptions, pressure feeling, as well as motor system activities depending on sensory input (e.g., Parkinsons, myocionias, dystonlas, tremor, stiff man syndrome, dyskinesia, tremor, dystonia, chorea and ballism, tic syndromes, Tourette's Syndrome, myoclonus, drug-induced movement disorders, Wilson's Disease, Paroxysmal Dyskinesias, Stiff Man Syndrome and Akinetic-Ridgid Syndromes and Parkinsonism, etc.). Pain can include chronic pain, acute pain or subacute pain.

Peripheral neuronal tissue. Peripheral neuronal tissue refers to any neuronal tissue associated with a nerve root, root ganglion, or peripheral nerve that is outside the brain and the spinal cord, including the autonomous nervous system, inclusive of (ortho) sympathetic and parasympathetic systems, and thus the term non-peripheral neuronal tissues excludes these categories.

Phase difference. a difference in phase between two stimulation waves. Waves may be generally in phase (having a phase difference of zero, or approximately zero), generally out of phase (having a phase difference 180 degrees, or approximately 180 degrees), or have some other intermediary phase difference.

Psychiatric condition or disorder. A behavioral or mental pattern that causes significant distress or impairment of personal function; it can be used herein interchangeably with the term psychological disorder or mental disorder.

Psychological condition or disorder, a psychological pattern associated with distress or disability that occurs in a person and is not a part of normal development or culture. It can be used interchangeably with term psychiatric disorder or mental disorder.

Primary somatosensory cortex. This term refers to the brain region located in the postcentral gyrus and in the posterior part of the paracentral lobule. The primary somatosensory cortex also includes Brodmann areas 3, 1 and 2.

Proximate. As used herein, the terra “proximate” means on, in, adjacent, or near. Thus, to be proximate, one or more of the electrodes on an electrical stimulation lead are adapted to be positioned on, in, adjacent, or near the identified target tissue in the brain.

Secondary somatosensory cortex. “Secondary somatosensory cortex” refers to the brain region that lies ventral to the primary somatosensory cortex area along the superior bank of the lateral sulcus.

Slow wave: a waveform (herein a brainwave) with frequency band falling into a range of from about 0.1 Hz to about 1 Hz.

Somatosensory cortex or sensory cortex. As used herein, the term “somatosensory cortex” or “sensory cortex” includes the primary somatosensory cortex, secondary somatosensory cortex and the somatosensory association cortex, as well as the Brodmann areas associated therewith, Still further, the sensory cortex includes all cortical sites having projections to or from the sensory cortex, as well as the subcortical sites having projections to or from the sensory cortex.

Spike. “spike” refers to an action potential. Yet further, a “burst spike” refers to a spike that is preceded or followed by another spike within a short time interval (Matveev, 2000), in other words, there is an inter-spike interval, in which this interval is generally about 100 ms but can be shorter or longer, for example 0.5 milliseconds. A spike in a burst may be used interchangeably with the term “pulse”.

Stimulation or stimulation wave. Stimulation, or to stimulate, as used herein, refers to chemical, electrical, magnetic, optical, sound, thermal and/or another such stimulation that modulates the predetermined neuronal sites; in this application, this shall largely refer to neurostimulation. Where there is a plurality of stimulation waves, there may be phase differences from instantaneous phase synchronization to up to 359 degrees of phase lag. Phase differences may be defined in terms of: phase synchronization, which may be used interchangeably with instantaneous phase synchronization plus lagged phase synchronization, which is not instantaneous, but does have a delay; activity within a network, which is instantaneous, more or less; or activity between networks, in which instantaneous or anticorrelated or different phases may exist, for example at 90 degrees or at 270 degrees. Note also that different waveforms may be applied to different networks. Stimulation design may be adjustable in an automated, closed loop manner through the ability of the system to have designed within it a sensing functionality of the recognition of the presence and/or magnitude of correlated and anticorrelated activity.

Sub-acute pain. As used herein, the term “sub-acute pain” refers to slow, insidious onset of pain, which can also be characterized as dull and achy. At times, sub-acute pain might not be easily localized, however, it may be possible to localize the pain depending upon the condition. Typically, sub-acute pain creates a discomfort for the person, but does not typically impair functionality for the person.

Theta. Theta is a brainwave frequency band falling into the range of from about 4 Hz to about 8 Hz.

Tissue in the brain. As used herein, the term “tissue in the brain” includes any tissue in any way associated with the brain, including gray matter and white matter that make up the brain.

Tonic Firing or Tonic Mode. “Tonic firing” or “tonic mode” refers to a sustained response, which activates during the course of the stimulus, as opposed to phasic firing, which refers to a transient response with one or few action potentials at the onset of stimulus followed by accommodation.

Transcranial Pulse Stimulation (TPS). A non-invasive neurostimulation method that uses pulses delivered to the brain from a source external to the cranium.

Treating and Treatment. “Treating” and “treatment” refer to modulating predetermined neuronal sites, particularly central neuronal tissue, so that the subject has an improvement in the disease or condition, for example, beneficial or desired clinical results. For purposes of this invention, beneficial or desired clinical results include, but are not limited to, alleviation of symptoms, diminishment of extent of disease, stabilized (e.g., not worsening) state of disease, delay or slowing of disease progression, amelioration or palliation of the disease state, and remission (whether partial or total), whether detectable or undetectable. One of skill in the art realizes that a treatment may improve the disease condition but may not be a complete cure for the disease, which will be unpredictable and unknowable until successful animal model experiments and successful clinical testing and observation of a novel treatment have been completed, and that this is a process that is entirely unpredictable and has no reasonable expectation of success without experimental observation and verification. One of ordinary skill in the art may have an understanding of those methods of clinical interventions and/or of those electrical or electronic devices that may more quickly lead to a discovery or an invention, but again, no one knows for certain whether administration to a patient will be safe and efficacious until an adequately well-designed, reviewed, conducted, observed, and reported trial has been completed.

Action Potentials and Their Propagation. Information is conveyed through the nervous system via neuronal cells along their membranes and across synaptic junctions. Thus, the neuronal cells process information by both passive processes (e.g., electrical properties of the membrane which enable spatial and temporal summation) and active processes (e.g., propagation of the action potential, signal amplification or attenuation, and synaptic transmission). Generation of an action potential at the axon initial segment requires passive summation of multiple inputs, as well as signal amplification before membrane depolarization reaches threshold, thus the passive and active processes are interdependent.

The generation of the action potential initially depends upon the electrical properties of the cell. It is known that cells have an electrical voltage difference across their membranes, the membrane potential. Several types of protein pores or ion channels are responsible for maintaining and altering the membrane potential of the cell. Voltage-gated sodium channels, which have a low threshold, are responsible for the explosive depolarization of the membrane potential that forms the action potential or spike, whereas, the voltage-gated potassium channels are responsible for the repolarization of the membrane potential. For excitation, stimulatory input results in a net increase in the inward flow of sodium ions compared to an outward flow of potassium ions, which results in a depolarizing cell membrane potential change. For inhibitory inputs, potassium and chloride ion channels are opened which drives the membrane potential away from threshold (hyperpolarization). As one of skill in the art realizes, neurons receive multiple excitatory and inhibitory inputs, thus a summation of these inputs occurs, for example temporal and spatial summations. Temporal summation occurs when a series of subthreshold impulses in one excitatory fiber produces an action potential in a postsynaptic cell. Spatial summation occurs when subthreshold impulses from two or more different fibers trigger an action potential.

Once the initial action potential is generated, the information is conveyed via axonal conduction or synaptic transmission (e.g., chemical or electrical). Electrical synapses are found not only in the brain, but in heart and smooth muscle and epithelial liver cells. However, in the brain, electrical synapses (also known as gap junctions) are less common than chemical synapses, and are characterized by rapid speed of transmission and do not readily allow inhibitory actions or long-lasting changes in effectiveness. Gap junctions allow the bidirectional passage of not only ions, but other small molecules. In humans, astrocytes also contain gap junctions to mediate potassium buffering.

Chemical synapses, on the contrary, do mediate either excitatory or inhibitory actions. Another difference between chemical and electrical transmission is that electrical can be bidirectional since the ion channels connect the cytoplasm of the presynaptic and the postsynaptic cells, whereas chemical transmission is typically unidirectional since there is no continuity between the cells. Chemical synapses comprise a presynaptic element that contain vesicles comprising neurotransmitters and a postsynaptic element which contains receptors for the neurotransmitters. Transmitter release is initiated when the nerve terminal is depolarized by an action potential resulting in a rapid influx of calcium ions into the nerve terminal. This rapid influx of calcium ions causes a fusion of the vesicles to the presynaptic membrane, and ultimately the release of the neurotransmitters, which then bind to their receptor located on the postsynaptic membrane.

The ability of the neuronal cell to fire or produce action potentials may vary depending upon its biophysical properties (e.g., types of ionic channels, etc.) and/or its position in the circuit or nervous system. Thus, cells can respond to an input (stimulatory or inhibitory) with a decelerating train of action potentials, an accelerating train of action potentials or a constant firing frequency. For example, an increase in firing of a neuronal cell may be a result from increased amounts of calcium ions or a function of residual increase in calcium ions left over from the first stimulation (also known as facilitation) in the presynaptic element, which results in an increased release of the neurotransmitter. Thus, a second stimulation can occur within milliseconds of the first. Conversely, a second stimulation may result in inhibition and not facilitation of the response if an inhibitory interneuron is activated, which feedbacks to the first neuronal cell to inhibit firing.

### Brain Network Connectivity and Stimulation Patterns.

Default Mode Network (DMN). The default mode network (DMN) is a network of interacting brain regions that is active when a person is not focused on the person's outside world, and is measureable by using the fMRI technique. In the field of neuroscience, the DMN may also be known as the default network, the default state network, or anatomically as the medial frontoparietal network (M-FPN), and is generally described as a large-scale brain network that is primarily composed of the medial prefrontal cortex, the posterior cingulate cortex/precuneus, or the angular gyrus. The DMN is best known for being active when a person is not focused on the outside world and the brain is at wakeful rest, such as during daydreaming and mind-wandering. It may also be active during detailed internally directed thoughts related to external task performance. Other times that the DMN is active include such times as when the individual is thinking about others, thinking about themselves, remembering the past, or planning for the future. The DMN was originally noticed to be deactivated in certain goal-oriented tasks and was sometimes referred to as the task-negative network, contrasting with the task-positive network. These nomenclature are not preferred, because it is now known that the network can be active in internal goal-oriented and conceptual cognitive tasks. The DMN has been shown to be negatively correlated with other networks in the brain such as attention networks the CEN and commonly the salience network.

The brain, however is constantly busy and does not cease activity when a subject is at rest. Metabolism in the brain stays the same when a person goes from a resting state to performing math problems requiring mental effort, suggesting that active metabolism in the brain must also be happening during rest. In fact, the brain's energy consumption is increased by less than 5% of its baseline energy consumption while performing a focused mental task. This shows that the brain is constantly active with a high level of activity even when the person is not engaged in focused mental work. Raichle coined the term “default mode” in 2001 to describe resting state brain function; the concept rapidly became a central theme in neuroscience. Around this time the idea was developed that this network of brain areas is involved in internally directed thoughts and is suspended during specific goal-directed behaviors. In 2003, Greicius and colleagues examined resting state fMRI scans and looked at how correlated different sections in the brain are to each other, creating correlation maps. Since then other networks have been identified, such as visual, auditory, and attention networks, and some of them are often anti-correlated with the default mode network.

The default mode network is thought to be involved in several different functions. It is potentially the central nervous system basis for the self, when it engages in autobiographical information, i.e. memories of collection of events and facts about oneself; self-reference, that is, referring to traits and descriptions of one's self; and emotion of one's self, which is the practice of reflecting about one's own emotional state.

It is potentially the neurological basis for thinking about others, including the theory of mind-thinking about the thoughts of others and what they might or might not know; the emotions of others, in understanding the emotions of other people and empathizing with their feelings; in moral reasoning, determining a just and an unjust result of an action; social evaluations for example forming good-bad attitude judgments about social concepts; in social categories, as in reflecting on important social characteristics and status of a group; and in social isolation, such as a perceived lack of social interaction. And, it is potentially the neurological basis of remembering the past and thinking about the future, including remembering the past; imagining the future; envisioning events that might happen in the future; episodic memory, that is, detailed memory related to specific events in time; story comprehension such as understanding and remembering a narrative; and replay, here meaning consolidating recently acquired memory traces. Additionally, during attention demanding tasks, sufficient deactivation of the default mode network at the time of memory encoding has been shown to result in more successful long-term memory consolidation. Studies have shown that when people watch a movie, listen to a story, or read a story, their DMNs are highly correlated with each other. DMNs are not correlated if the stories are scrambled or are in a language the person does not understand, suggesting that the network is highly involved in the comprehension and the subsequent memory formation of that story. The DMN is shown to even be correlated if the same story is presented to different people in different languages, further suggesting the DMN is truly involved in the comprehension aspect of the story and not the auditory or language aspect.

In accordance with embodiments disclosed herein, optimal targets within the nervous system are selected for neuromodulation. The optimal targets are selected according to network connectivity within the nervous system of a patient. For example, the brain of a patient may be modeled as a complex adaptive system of one or more neural networks. The brain may be viewed as exhibiting small world topology characteristics. That is, the brain functions as a modular scale free hierarchical network (e.g., fractal in organization). Also, the brain functions in the presence of noise (equivalently variability in neural activity) see, for example U.S. Pat. No. 8,682,441 in the field of pink noise therapy, the entire disclosure of which is incorporated herein by reference. In a noisy, hierarchical organization, the brain functions as a complex adaptive network of interconnected modules. By selecting one or more nodes within one or more networks within the brain for stimulation, a central nervous system disorder may be treated by strengthening or weakening the network connectivity, by controlling the phase of signals that pass along networks, or by creating correlated or anticorrelated connectivity communication relations between networks of interest to treat an identified central nervous system disorder.

Certain connectivity between neural populations in the brain may be defined by structural connectivity. The structural connectivity may be determined in studies using diffusion tensor imaging (DTI), diffusion spectrum imaging (DSI) or diffusion kurtosis imaging (DKI) as examples. Connectivity may also be the result of functional connectivity in a network. The functional connectivity may be determined by correlation or anticorrelation in neural activity in one or more respective brain areas or brain networks. Also, connectivity may be related to effective connectivity, which can be considered directional functional connectivity, through the result of information transfer between neural nodes and networks.

Any number of suitable mechanisms may be employed to measure neuronal activity for suitable processing. For example, EEG (or electroencephalogram) is a recording of brainwave activity. QEEG (Quantitative EEG), popularly known as brain mapping, refers to a comprehensive analysis of brainwave frequency bandwidths that make up the raw EEG. QEEG is recorded the same way as EEG, but the data acquired in the recording are digital and not analog, and can therefore be used to create topographic color-coded maps that show electrical activity of the cerebral cortex. In an QEEG analysis, the electrical activity of the brain is measured by placing a number of electrodes or sensors about the head of a patient and the sensors are connected to a recording device. Electrical activity is recorded using the sensors for typically five to thirty minutes. The data representing the recorded electrical activity is suitably processed. The processing provides complex analysis of brainwave characteristics such as symmetry, phase, coherence, amplitude, power and dominant frequency. Such processing enables the correlation, coherence, and relevant activity metrics indicative of functional connection between brain locations to be identified. The analysis enables activity falling above or below a statistical norm to be identified for locations within the brain. Also, the activity may identify activity above or below the norm for relevant brainwave frequency bands (delta, theta, alpha, beta, and gamma bands as examples). The activity variance from the norm can be expressed relative to a calculated standard deviation of activity data. Further, the QEEG analysis additionally enables functional connectivity to be identified by frequency, amplitude or phase coherence analysis or a combination hereof, eg phase-amplitude cross-frequency coupling, of activity between different neural sites. A specific form of functional connectivity evaluates the phase relationship of infraslow or slow frequencies, also known as correlated or anticorrelated activity. This is especially described by fMRI, but can also be detected by EEG, MEG, PET scan or other brain imaging techniques. The functional connectivity can be likewise expressed in terms of above or below the norm relative to a standard deviation calculation. Additional and/or alternative processing of recordings of electrical activity in the brain of a patient may be employed to assist identification of variations in functional connectivity related to a central nervous system disorder according to some embodiments. For example, QEEG combined with LORETA (Low Resolution Electromagnetic Tomography) enables examining of deep structures of the brain slice by slice, as well as viewing 3-dimensional models of the brain and may provide a suitable analysis to identify functional connectivity resulting from a central nervous system disorder to be treated according to representative embodiments. Also, the BrainWave® software application (available from the Department of Clinical Neurophysiology, VU University Medical Center, Amsterdam, The Netherlands) is an application for the analysis of multivariate neurophysiological data sets (such as EEG data sets). The BrainWave® application provides several measures of functional connectivity (coherence, phase coherence, imaginary coherence, PLI and synchronization likelihood) among other relevant neural activity metrics. The functional connectivity mapping of the BrainWave® application may be employed to assist identification of variations in functional connectivity related to a central nervous system disorder in a patient according to some embodiments. In an alternative embodiment of the invention, a network representation of neural activity is created using graph and network concepts.

Improper connectivity from a neurological disorder is addressed according to embodiments disclosed herein. In some embodiments, insufficient or decreased functional and/or effective connectivity related to a neurological disorder is strengthened by simultaneous or otherwise synchronized stimulation in two or more nodes of one or more neural networks relevant to the neurological disorder. In some embodiments, excessive functional and/or effective connectivity related to a neurological disorder is weakened by defective, malfunctioning, or unwanted correlated or anticorrelated communication connectivities between identified functional nodes or networks, along one or more neural pathways of a neural network relevant to the respective neurological disorder. In a network connectivity framework, the centrality of a node refers to how many of the shortest paths between all other node pairs in a network pass through the respective node. As discussed herein, a hub refers to a network node in a neurological network which exhibits a high degree of centrality. Neurological hubs connect to many other brain areas. “Rich club” neurological sites refers to neurological sites that are hubs and are connected to many other hubs. Rich club sites integrate neurological activity from different networks and different neurological modules.

According to some embodiments, sites for neuromodulation are selected according to identified hubs (for example feeder hubs or hubs of the rich club or core) By selecting hub and rich club sites, improper connectivity (associated with a given neurological disorder) can be more effectively strengthened or weakened depending upon the specific neurological disorder.

Multiple types of stimulation patterns may be employed for network stimulation according to respective embodiments including tonic, burst, noise stimulation patterns, infraslow and nested infraslow patterns, as examples.

In some embodiments, nested stimulation may be provided to one or more nodes within one or more neural networks in association with adjusting connectivity within or between one or more networks. Details regarding design and generation of nested stimulation may also be found in U.S. Pat. No. 10,076,668 entitled “System And Method For Nested Neurostimulation,” the entire disclosure of which is incorporated herein by reference.

Colors of Noise. Grey noise is a combination of pink noise and blue noise, or of brown noise and purple noise, or of black noise and blue noise, or of black noise and purple noise. The relationships between categories of noise signals, their beta, or brainwave neural oscillation values, and real-world characterizations is given in the following two tables.

| | | | | |
|---|---|---|---|---|
| Color | Black | Brown, equal | Pink | White |
| | | to Red | | |
| 1/f^{α} | α > 2 | α ≈ 2 | α ≈ 1 | α ≈ 0 |
| What | Natural | Random walk | Nature | Pure Noise |
| | disasters | | | |

| | | | | |
|---|---|---|---|---|
| Color | Blue, equal to | Violet, equal | Grey | Green |
| | Azure | to Purple | | |
| 1/f^{α} | α ≈ −1 | α ≈ −2 | U-Curve | Pink, with an |
| | | | | additional 500 Hz |
| What | Dithering | Acoustic | Perceived | Background noise |
| | | thermal noise | as equal | of world |

Firing Modes. Different firing modes or frequencies occur in the brain and/or other neuronal tissue, for example tonic firing and burst firing (including both irregular or regular burst firing), The thalamus for example utilizes both types of firing modes. The two thalami (bilateral paired structures) are the gateways to the cerebral cortex and, thus, to consciousness. The thalamic nuclei specialize in several different signaling functions: transmitting signals from sensory input to the cortex; transmitting signals from cortical motor centers to effectors; transmitting control signals that select which input and output will be permitted to pass to and from the cortex and how the signals will be sequenced (thalamic reticular nuclei (TRN)); modulated (controlling intensity), synchronized, or grouped (Interlaminar Nuclei (ILN)).

All thalamic relay neurons pass through the TRN, which opens and closes their “gates” going to the cortex, (McAlonan and Brown, 2002). One mode that TRN neurons use to transmit these relays is burst firing mode. This mode is useful for activating a small population of neurons in the cortex for a short period. In contrast, the continuous (tonic) firing mode permits a thalamic neuron to transmit a steady stream of signals to the cortex. The tonic firing pattern triggers looping activation in the cortical circuits that receive the signals. Evoking looping, or “recurrent” activation in the cortex requires a steady neural input.

Tonic or burst firing mode may be related to the molecules which are associated with the neurons. Such molecules include either parvalbumin (an egg-derived protein also a calcium-binding protein) or calbindin (a calcium-binding protein). Tonic firing is found especially in cells that contain parvalbumin. It behaves in a linear fashion, for example, the auditory thalamus (MGBV) fires at a specific frequency and the auditory cortex will follow at the same pace with a minor phase difference (Miller et al., 2001) of 2 ins. Tonic firing, however, can be overruled by burst firing (Lisman 1997: Sherman 2001; Swadlow and Gusev 2001).

Burst firing is typically found in calbindin positive cells (Kawaguchi and Kubota 1993; Hu et al., 1994; Hu 1995; He and Hu 2002). Thus, burst mode firing may utilize a calbindin system to generate the burst. Generally, burst firing is accomplished through the activation of either a subthreshold membrane conductance that initiates action potentials or a suprathreshold membrane conductance that once activated evokes two or more action potentials. Sodium (Na⁺) and calcium (Ca²⁺) activated conductances have all been implicated in burst generation.

Burst firing acts in a non-linear fashion with a summation effect of each spike, thus more readily activating a target cell than tonic firing can. Burst firing has been described in drowsiness, slow wave sleep, and anesthesia, as well as epilepsy in the thalamus. Neural network modeling has further demonstrated that bursts are generated by positive feedback through excitatory connections. In networks of two populations, one excitatory and one inhibitory, decreasing the inhibitory feedback can cause the network to switch from a tonically active, asynchronous state to the synchronized bursting state.

The generation of repetitive burst discharges in neurons is correlated with the generation of gamma frequency (30-80 Hz) oscillations in the local field potential. It is believed that conscious perception depends on gamma band frequency activity.

Increasing depolarization to hyperpolarization induces a prolonged refractory period of tonic firing resulting in single spike bursts (for example in the visual system), and further depolarization results in progressively more spikes per burst. Further depolarization will temporarily silence the cell.

It is hypothesized according to the present invention that burst stimulation may be used by neuronal tissue to process information in a manner that is similar to amplitude modulation. Specifically, the spacing between individual bursts in a burst stimulus may be used to signal information to various regions in the brain. That is, the spacing between the bursts can vary (hence are “amplitude modulated”) to convey information. The signaled information can be related to relevance information. The signaled information could also be related to signaling the beginning and ending of certain packets of information. By providing electrical burst stimulation from an implantable pulse generator, which is one alternative embodiment of the present invention, it is possible that the stimulated brain tissue will change its processing of other stimulus information. For example, by appropriately selecting an interburst interval, auditory information that would otherwise be problematic to a patient could become ignored by a respective segment of the brain due to a lack of “relevance” and/or a lack of synchronization with the arrival of the burst stimulus.

Nested Stimulation and Electrical Stimulation Devices. Neurological stimulation (NS) systems, designated herein as implantable pulse generators, execute the function of electrically stimulating a predetermined site area to treat one or more neuropsychiatric disorders or conditions as part of the Brain Multi-Network Modulation therapeutic field of the present invention as claimed. In general terms, a pulse generator typically includes a pulse generating source that is surgically implantable for delivering the stimulation waves to the brain. This can be contrasted with non-invasive medical devices, which do not include components implanted into the patient's body and are not the subject of this application. The delivery system may deliver a designed pattern of electrical pulses to a predetermined site. In certain preferred embodiments, the delivery system may include one or more delivery devices each coupled directly to the connecting portion of a stimulation lead. In some embodiments, a delivery device is incorporated into a stimulation. For example, such a stimulation system may function in the manner as the technologies developed by Neuroelectrics or in the manner of High Definition transcranial electrical stimulation devices commercialized by other companies such as Soterix Medical, Nesstrim PLC, Smartfocus, Brainsway, or Neuroconn GmbH. Whether the delivery device is coupled directly to, or embedded within, the stimulation lead, the delivery device controls the stimulation pulses transmitted to one or more stimulation electrodes that are located on the stimulating portion of a stimulation lead, and is positioned so as to be in communication with a predetermined site, according to suitable therapy parameters, for example noise color, correlation anticorrelation, phase coordination, phase non-coordination, duration, amplitude or intensity, frequency, pulse width, firing delay, and the like.

Central nervous system stimulation (NS) systems, designated herein as an implantable stimulator, execute the function of electrically, magnetically, acoustically, or optically stimulating a predetermined site area to treat one or more central nervous system disorders or conditions as part of the Multiple Network Modulation therapeutic field within which the present invention as claimed operates. In general terms, a puse generator typically includes an implantable pulse generating source or electrical IMD, (generally referred to as an “implantable medical device” or “IMD”), and further includes one or more implantable electrodes or electrical stimulation leads for applying a designed pattern of electrical pulses to a predetermined site. In operation, both of these primary components may be implanted in the person's body, as discussed below, and which is one embodiment of the present invention. In certain preferred embodiments, an IMD is coupled directly to the connecting portion of a stimulation lead. In some embodiments, an IMD is incorporated into a stimulation lead and the IMD is instead embedded within the stimulation lead. For example, such a stimulation system may be commercially obtained as the Bion® stimulation system manufactured by Advanced Bionics Corporation. Whether the IMD is coupled directly to, or embedded within, the stimulation lead, the IMD controls the stimulation pulses transmitted to one or more stimulation electrodes that are located on the stimulating portion of a stimulation lead, and is positioned so as to be in communication with a predetermined site, according to suitable therapy parameters, for example noise color, correlation anticorrelation, phase coordination, phase non-coordination, duration, amplitude or intensity, frequency, pulse width, firing delay, and the like.

A System of Brain Network Modulation Devices. As stated above, current techniques in brain stimulation are still largely based on a phrenological approach that a single brain target can treat a brain disorder. Even though a single target can modulate an entire network, research in network science reveals that many brain disorders are the consequence of maladaptive interactions between multiple networks rather than a single network. Consequently, targeting the main connector hubs of those multiple interacting networks involved in a brain disorder is of significantly greater beneficial breadth of design for the targeting of the vast array of neurological, psychiatric, and psychological disorders that may present in the clinic. We have thus conceived of a next generation of a systematic approach to the design and implementation of a network of implants, that are the subject of the present invention, which will rely on distributed, multisite neuromodulation to effect change in the brain, offering more precise and localized stimulation to targeted interacting brain networks. With the potential to enhance the effectiveness of targeting multiple areas of the interacting networks, this concept holds great promise as a universal approach to treat neurological psychiatric, and psychological disorders.

It has become evident that most brain disorders, whether neurological, psychiatric, or psychological in nature, are not the consequence of a phrenological hyperactivity of one disease-provoking area in the brain, but rather comprise the emergent properties of altered inter-area and intra-area network activity and connectivity These normal interactions within and between networks can become altered from the normal baseline and become pathological, resulting in brain disorders. These disorder-related networks can be related to a decrease or increase in existing connections, a change in correlated or anticorrelated activity, or to new connections.

Psychosurgery, meaning the application of surgical lesions in the brain, was developed in the 1930s in an attempt to develop a more humane treatment for psychiatric disorders than being locked away for the rest of one's life in overcrowded and underfunded asylums. Intriguingly however, the outcome in hundreds of thousands of patients who underwent this form of treatment converged on a rule of three: ⅓ of patients were markedly improved, ⅓ were unaltered or slightly better, and ⅓ were unchanged or worsened. Once medication was discovered that could treat psychiatric disorders, then medication largely replaced psychosurgery. However, since around 2010 there has been a noticeable, steady decline of pharmaceutical industry interest in developing neuropharmacological products, whether for neurological, psychiatric, psychological indications. The economic reason is that developing medication for the central nervous system (CNS) has approximately a 50% lesser chance of making it to the market (6.2% vs 13.3%), takes approximately 30% longer (19.3 vs 14.7 months) and costs approximately 30% more than, for example, a heart medication. Consequently, large multinational biopharma companies have largely lost interest in neuroscience, resulting in a 50% decline in budgeted investment for brain-related diseases Neuromodulation can fill this therapeutic gap, but to do so will require a whole new breed of devices.

To illustrate, even though deep-brain stimulation is heralded as being highly successful, its success is relative; meta-analyses that evaluate the outcome of brain stimulation via implanted devices yield a 50% success rate, meaning that a 50% improvement in 50% of patients after 5 years is observed in the clinic. This success rate holds for every brain disorder for which implants are provided, be it depression, pain, tinnitus, OCD, dystonia, or Parkinson's disease, for example. Further, if outcome measures are recorded in a non-binary way (that is, of responders versus non-responders), but in a way that allows for 3 possible outcomes, this results in an observed outcome of ⅓ showing major improvement, ⅓ showing somewhat of an improvement and ⅓ not showing an improvement or showing a worsened condition, which is similar to the observed outcomes that are noted in psychosurgery. Thus, there is a large margin for improvement and a clear long-felt but unmet need for improvement. In the setting of Parkinson's disease, for example, it has been argued that these unsatisfactory long-term results are not to be seen as failures of the DBS procedure per se but are resultant from further progression of a degenerative disease. However, this argument is clinically unconvincing, since outcomes in pain, tinnitus, major depression etc. are similar, yet these disorders have not been considered as degenerative brain pathologies.

From an evolutionary biology point of view, the brain has evolved to reduce the inherent uncertainty that is ever-present in a changing environment, especially since the appearance of mobile living creatures in the world. Viewed this way, the brain can be seen as a Helmholtzian prediction machine that actively and constantly samples the internal and external environment of the organism for information to update its calculated predictions in a Bayesian manner.

From an engineering point of view, the brain is a complex adaptive system that is analogous to the internet, or an ant colony, or the economy, or to a social-relations network. A complex adaptive system can be interchangeably used with the terms complex dynamic system, and in the setting of the brain, as a complex neuroplastic system. A complex adaptive system has an intermediate topology between two extremes, namely a lattice-like regular topology at one end of a spectrum, and a random topology at the other end. Existence at the extremes is not compatible with conscious brain states and those areas are not adaptive, since a lattice topology is fully determined, while a random topology is completely free. Importantly however, a complex adaptive system has a small world topology and is characterized by adaptive flexibility, i.e. it is amenable to neuromodulation. For the brain to qualify as a complex adaptive system, it must be able to fulfill two criteria. Firstly, it requires a structure following a ‘small-world topology’ and secondly it has to embed noise. These two characteristics permit a system to be adaptive and flexible.

The brain is noisy but that noise is structured, and it generally follows a 1/f or 1/f² power-law distribution. The 1/f structure implies that a network has memory, and can carry information, in contrast to white noise, which reflects pure randomness (see paragraph [0132] above, the colors of noise). A system with a power-law distribution can learn, while still maintaining stability. All complex adaptive systems share the same characteristics, one of which is ‘emergence’, meaning that the whole is more than the sum of its components. Thus, from its individual components, its combined network properties cannot be predicted; the properties must emerge to be observed. Thus, emergence is a process whereby larger entities, patterns, and regularities arise through interactions among smaller or simpler entities that themselves do not exhibit such properties. A collection of all of the constituent parts of a car do not make a car, unless they are connected in a very specific way that permits a functional car to emerge from the parts. Of course, a standard car is not adaptive; it is a complex system, but it is not adaptive. Although it is adaptive, in a complex adaptive system like the brain, not every adaptation is beneficial, and maladaptive changes can lead to neurological, psychiatric, or psychological disorders, caused by maladaptive activity and/or connectivity changes.

Based on this concept, many brain disorders have indeed been regarded as connectivity problems, and consequently network science has been embraced as a novel approach for studying brain disorders. Connectivity in a complex adaptive system that exists at an anatomical level is called structural connectivity, and at a functional level, is called functional and effective (i.e., directional-functional) connectivity. Structural connectivity refers to the presence of anatomical, biological-fiber pathways in the nervous system, which are relatively static at shorter time scales (seconds to minutes) but can be dynamic at longer time scales (hours to days) during learning or development. Thus, even anatomical connections are not hardwired, but change with experience or deprivation thereof. Functional connectivity on the other hand, is fundamentally a statistical and not an anatomical concept, looking at patterns of correlated activity between different brain areas by measuring frequency or phase. In contrast to structural connectivity, which is based on hardwired anatomical white-matter tracts, functional connectivity changes constantly, by a process of instantaneously adjusting correlated activity to either endogenous or exogenous stimuli. Another form of functional connectivity computes cross-frequency coupling between different oscillatory frequencies, in which higher oscillations (i.e. beta and gamma) are nested in a hierarchical manner on slower oscillatory frequencies (infraslow, slow, delta, theta and alpha), which act as carrier waves. Functional connectivity does not assume any directional flow of information. This is implicitly calculated by effective connectivity, which computes the flow of information from locus to locus in the brain. Effective connectivity can, therefore, be considered a directional functional connectivity, and is often based on a time series, where the underlying idea is that causes will predate effects. Structural, functional and effective connectivity are all thus related to each other.

Functional connectivity is the basis of multiple separable brain networks, yet these brain networks are not all active at the same time. When one network is activated, others may be inactive or less active, resulting in anti-correlated activity between these networks. Some separable networks may be co-activated, leading to correlated activity. This has led to the development of the triple network model, which is a network science-based approach explaining core interactions in multiple cognitive and affective disorders. It states that neurological, psychiatric, and psychological disorders are the result of aberrant interactions within and between three canonical brain networks. These three networks include the self-representational default mode network, the behavioral relevance encoding salience network, and the goal oriented frontoparietal central executive network. Normally, the salience network and the central executive network are characterized by correlated activity, and both networks are anti-correlated to the default mode network. The salience network acts as a switch between the anticorrelated default mode network and the central executive network. This is in keeping with the proposed functions of the three networks. When the salience network identifies a behaviorally relevant event in the environment, it reduces the activity of the self-oriented and ‘mind wandering’ default mode network and activates the external goal-oriented central executive network to deal with the external salient event. Functional and effective connectivity is constrained by the presence of both direct and indirect anatomical connections, and correlated activity can change structural connectivity via Hebbian mechanisms (cells that fire together wire together). These dynamical changes in structural, functional and effective connectivity are the basis of the concept of neuroplasticity and are crucial to developing novel devices that can not only break pathological connections but also replace and rebuild normal physiological connections. It has been proposed that this requires two different stimulation designs, one that can optimally strengthen connectivity, such as a burst-like stimulation, and one that can break functional connections, such as a noise-like stimulation. Hyperconnectivity may be treated by the surgical cutting of the target connection or electrophysiologically by noise stimulation, low frequency stimulation in the antiphase, or by pseudorandom burst stimulation. Hypoconnectivity, on the other hand can be treated by burst stimulation in two targets in synchrony, by infraslow frequencies in phase stimulation, and by noise stimulation. Noise stimulation can thus both break and build connectivity via a stochastic resonance effect. Multiple brain disorders exhibit similar changes in network activity and connectivity. These common pathophysiological mechanisms can be both genetic, physiological and anatomical.

The same risk genes may cause multiple different neurological and psychiatric disorders, known as pleiotropy. Depending on the environment, the same risk genes may change functional connectivity, by modulating epigenetic gene expression in the brain, resulting in different emergent properties, i.e. different neurological and psychiatric disorders. For example, genetic overlap exists in the reward deficiency syndrome, a group of disorders encompassing addictions (substance and non-substance), impulsivity, obsessive compulsive and personality disorders with a common underlying mechanism.

Electrophysiologically, the entity called thalamocortical dysrhythmia groups pain, tinnitus, Parkinson's disease, depression and slow-wave epilepsy, and is characterized by a common core of beta activity in the dorsal anterior cingulate cortex (dACC) and the parahippocampus, and theta-gamma or theta-beta cross-frequency coupling in the respective motor or sensory cortex distinguishing the separate clinical entities.

Furthermore, many psychiatric disorders (schizophrenia, bipolar disorder, depression, addiction, obsessive-compulsive disorder, anxiety) share a common anatomical substrate. Salience-network dysfunction is at the core of these disorders. As the salience network, which is atrophic in many psychiatric disorders is dysfunctional, its function as a switch between internally directed cognition of the default mode network and externally directed cognition of the central executive network is disrupted. This leads to abnormal functional connectivity within and between these three networks as expressed by correlated and anti-correlated activity within and between the three cardinal networks. And indeed, common or shared hypo- and hyperconnectivity changes are identified in numerous brain disorders including ADHD, anxiety, depression, bipolar disorder, autism, OCD, PTSD and schizophrenia.

These anatomical and physiological shared mechanisms have led to our conception of the development of a set or series of universal brain network neuromodulators that target the common pathophysiological mechanisms of these pathologies, rather than developing a dedicated device for each disorder individually.

As an example, triple network neuromodulation to treat ADHD, anxiety, depression, bipolar disorder, autism, OCD, PTSD, or schizophrenia would involve sensing correlated activity within each of the 3 canonical networks, as well as between the 3 networks. If an abnormal infraslow phase synchronization is detected between some nodes, the nodes need to adjust their activity so that the intranetwork phase synchrony is restored, but also in such a way that the internetwork infraslow phase synchronies are restored to normal correlated activity between salience network and central executive network, and anticorrelated between these two networks and the default mode network. This cannot be achieved by single target or even dual target stimulation, but rather requires the integrated activity of multiple widely distributed stimulators, that can sense, communicate and stimulate in an adaptive and flexible way.

Using network science, which studies complex adaptive systems, it has been shown that random attacks on (brain) networks are not capable of disrupting a network, and thus are also not eliminating the emergent property of the network. Therefore, a targeted attack on the main hubs of the network or multiple interacting networks that are involved in the brain disorder is more likely to exert a beneficial effect. This conception is in agreement with a meta-analysis on deep brain stimulation for pain, which demonstrates that multitarget implants yield better outcomes than single-target stimulation, especially if both lateral and descending pain-inhibitory pathways are jointly targeted. Similarly, multitarget modulation also seems to be of greater benefit for tinnitus than single-target stimulation, both non-invasively, and invasively, which is the most highly preferred embodiment of the present invention herein disclosed and claimed.

Based on our described and disclosed theoretical and clinical evidence, logic, inferences, and conclusions, we argue that a need exists for, and have conceived of, a new invention comprising a set and system of network neuromodulation dot electronic devices, which we collectively term NeuroDots™. We argue that if the field of clinical neuromodulation is to move to a new era of more successful treatment, then irrespective of the brain disorder under study or treatment, such neuromodulatory dots may successfully address this long-felt and unmet need in human disease. The system of neuromodulatory dots of the present invention will have to incorporate a set of criteria, both for hardware and software, that will permit normalization of pathological activity and connectivity. In summary, our neuromodulatory dots are multiple independent small stimulating and recording electronic devices that interact with each other (like ants in an ant colony) to deliver an adaptive set of synchronizing and/or desynchronzing stimuli, based on sensed data that measure activity and connectivity, calculated stimulatory output. The neuromodulatory dots all connect to a central control system via a cranial implant. In the following sections, we proceed to outline a list of clinical and technical requirements, features, details, and advantages, whose collective capabilities enable these therapeutic effects.

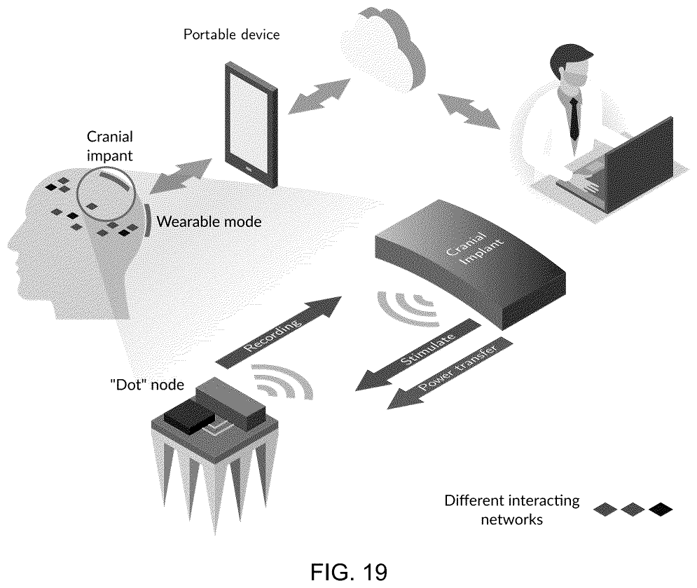

An overview of our conception of neuromodulatory dots is shown in FIG. 19, which depicts how the different implantable devices are connected to the clinician 1902 via wearable and portable devices 1904, 1906, 1910. We envision the neuromodulatory dots 1906 to constitute nodes 1906 that are designed and configured in such a way that they will be sufficiently powerful and diverse in function, ideally only requiring software updates, like apps on a smartphone, to target different brain disorders. Such a generic design approach has already been shown to be possible for traditional implantable devices, e.g., cardiac pacemakers. In the NeuroDots™ concept, multiple independent stimulating and recording devices 1904, 1906, interact with each other to receive and record data inputs and deliver an adaptive set of synchronizing or desynchronzing stimuli output signals 1905, based on sensed data that measure activity and connectivity. The neuromodulatory dots all connect to a central control system via a plurality of cranial implants 1908.

We teach multiple functional requirements for the neuromodulatory dots implants, from a clinical standpoint, described as in the following examples.

Multisite. Multiple, independently controllable but communicating small devices, small enough that they can be thought of and referred to as dots, are able to sense activity (local-field potentials) and connectivity with high resolution, i.e. frequency, amplitude and phase relationships between the different other neuromodulatory dots that are implanted into the patient. Depending on the application, the neuromodulatory dot devices will also be able to stimulate multiple sites in the brain.

Multifunction. Neuromodulatory dot devices can, individually and as a network, provide multiple stimulation signal or wave designs that can restore connectivity (e.g., synchronous burst, infraslow in phase, stochastic noise) or break connectivity (e.g., asynchronous burst, antiphase infraslow or high amplitude noise).

Highly autonomous. The dot devices adjust their flexible output based on what is sensed, in a closed-loop fashion, without human intervention or constant monitoring.

In-vivo reprogrammable. NeuroDots™ operation is upgradable on-site and in-vivo, via software updates.

Data-logging. The neuromodulatory dots system is capable of storing sufficiently large amounts of operational data so as to facilitate advanced data analysis, patient diagnosis and treatment.

Generic in use. The NeuroDots system is flexible enough for the implants to be inserted in any desired anatomical target of a pathological brain network, in such a way that it can address different neurological problems.

Minimally invasive. The neuromodulatory dots system is as minimally invasive as possible for achieving high patient and provider user acceptability and lowest extent of tissue damage.

Chronic operation. The NeuroDots system is able to function for a very long time meaning in excess of 10 years.

Biocompatible. The NeuroDots components are compatible with the neighboring brain matter and circulating or migratory cellular bodies or solubilized molecules and tissues.

Ecological. Safe and reliable implantation, explantation and operation of NeuroDots components is an essential feature.

We teach multiple technical requirements for the neuromodulatory dots implants, from a clinical standpoint, described in the following examples.

Large area and volume coverage. Since the brain is a complex adaptive system, the neuromodulation dots devices either need to span a large area or volume of the brain or it needs to be subdivided into multiple distributed dot implants that are connected in a wired fashion, or wirelessly, all operating with a chosen and programmed degree of autonomy.

High elasticity & biostability. NeuroDots implants will be elastic, of similar elasticity as that of the tissue into which it/they is/are implanted. This is measured by the Young's modulus of elasticity. Since brain matter is extremely soft—as indicated by its very small Young's modulus of elasticity of only around 1.5 kPa, the brain implants themselves will have a similar elasticity and will furthermore be as soft as well, with similarly compatible bendability, flexibility, and stretch. Since brain tissue has certain movement capabilities, the dot implants need to be able to move along with the movements of the brain, following its curvature, along and amongst the gyri, sulci, and fissures of the brain lobes. This is in stark contrast to the prior art bulky and rigid implants in current clinical use, whose Young's moduli is frequently of around 150 GPa, which is, approximately eight orders of magnitude greater/stiffer than that of brain matter. Moreover, the electrodes that interface the electronics with the ionics of the brain, are usually organized in 1-dimensional arrays, with ring or semi-ring electrodes, or in planar (2D) arrays and thus limited in the volume that they can cover. The distribution of the NeuroDots™ functionality over multiple dot implants alleviate this volumetric requirement.

Efficacious neuromodulation. Efficacious neuromodulation is delivered by dot implants, which interact with neurons and glia by modulating their tendency to generate action potentials and thus may have an excitatory effect or an inhibitory effect on the neurons that they are connected to. The simplest approach is electrical stimulation, but other forms of neuronal and glial modulation may be achieved by magnetic, light or ultrasound stimulation. Electrical stimulation requires the electrodes to be in close vicinity of the neuromodulation site. Moreover, it requires a direct electrical contact between the electronics of the implant and the ionics of the tissue. Unlike electricity, magnetism-based neuromodulation does not require a direct contact but still requires close proximity to the neuromodulation site. Neuromodulation by means of light, often referred to as optogenetic neuromodulation, is a biological technique that uses the expression of lightsensitive ion channels, pumps or enzymes in the neurons, which can be achieved by means of gene therapy. Due to the limited optical transparency of the brain, in particular for shorter wavelengths, neuromodulation can only happen in the direct vicinity of the light source, and its power efficiency is relatively poor. Moreover, the integration of light sources into an implant imposes challenges on the encapsulation of the implant. Finally, the combination of gene therapy and implantation makes this neuromodulation technique less attractive for treatment purposes. A recent development is the use of ultrasound for modulation of neuronal activity. Ultrasound experiences little attenuation in the brain and can thus modulate neural activity at larger depths. Since the neuromodulation mechanism is indirect (neither electrical, nor chemical), its power efficiency is lower than that of electrical neuromodulation. Moreover, integration of ultrasound transducers into an implant is far from trivial. The network nature of the brain requires that, if modulation is applied at multiple sites simultaneously, this modulation happens with a well-controlled degree of synchronicity so as to have the best possible (network) response and (patient) outcome. If the neuromodulation sites are covered by more than a single implant, this requires careful orchestration of the timing of these implants and their waveforms.

High-resolution neural recording. Depending on the type of neurotransmitter being released and its connectivity in the network, neurons, once they fire, can have an excitatory or inhibitory effect, or no effect at all. To establish the desired effect on the network and thus its emergent property, the dot implants will also be equipped with high-density sensing/recording capabilities. The combination of neuro-modulation and -recording is required for closed-loop operation, via positive or negative feedback, and intelligent control. This may also help in reducing the power consumption and possible side-effects of the implants, as the neuromodulation can be optimized for the intended clinical application.

Wireless power transfer or energy harvesting. By definition, all active implants are electronic devices and, thus, will require electrical power to execute their functionality. This energy can either come from an internal energy source, such as a battery, or from an external energy source of which the energy is coupled into the implant. As a result of their limited energy density, batteries tend to be bulky in neuromodulation devices, which puts an upper limit on their use at multiple locations in the brain neuromodulatory network. Moreover, once near their end of life, they need to be replaced surgically, which might require a delicate surgical procedure if the implantation sites are deeper into the body or surrounded by delicate tissue. An more beneficial alternative embodiment is to use wireless power transfer, which can be carried out in either the electrical, magnetic, electromagnetic, optical or ultrasound domains. Electric or magnetic wireless-power transfer (WPT), also known as capacitive or inductive WPT, uses relatively large-sized elements (compared to other techniques) such as conductive plates and coils, and hence, is better suited for relatively large implants that are implanted superficially under the skin and, thus, can be in close contact with the external energy source. In the case of electromagnetic wireless power transfer, much of the power radiated by the transmitter is scattered around and, thus, only a fraction reaches the implant, especially when the implant is small and located deep in the tissue. Optical wireless power transfer is more suited for smaller implants, but, as mentioned above, suffers from the limited optical transparency of tissue, which decays exponentially with the distance between the transmitter and the receiver. It is therefore suited for shallow implants only that can be covered by a wearable transmitter. An exciting alternative is the use of ultrasound. As in the case for neuromodulation itself, the signal experiences little attenuation along the way. Moreover, it is particularly suited for tiny implants, such as the most preferred embodiment of the dot implants.

Wireless data transfer. Wireless data or information transfer, also known as wireless communication, is also crucial for driving the NeuroDots system. It requires either the generation of energy that is modulated by the information or the modulation of available energy according to the information. This latter principle is also known as backscattering or load modulation and is particularly suited for highly asymmetric communication, in which the implant is severely limited in resources (power and size) and the external device is not, but we find it to be less suitable for communication among implants in a network. The amount of data that can be communicated to and from the implant(s) per unit of time, the data rate, is linearly proportional to the available bandwidth of the communication channel and a received signal power and is inversely proportional to the amount of noise and interference received. Of the five energy domains mentioned above (electrical, magnetic, electromagnetic, optical and ultrasonic), due to the fundamental relation between bandwidth and carrier frequency on the one hand and power efficiency on the other hand, electrical, magnetic and ultrasonic wireless communication offer lower data rates than electromagnetic and optical power transfer. Higher data rates are particularly important in implants that have less autonomy and high demands on the number of recording and stimulation channels and on the accuracy and complexity of the waveforms acquired and generated for neuromodulation.

Homogeneous design. Dot implants are constructed that are homogeneous in structure and in functionality. Such homogeneity will not only allow for a drastic reduction of development costs and implantation effort but also for a robust, mix-and-match philosophy of deployment, where a single (or even a few) implants are not crucial to the correct functionality of the NeuroDots system since it will rely on a majority-based operation, by which it is meant that as long as enough implants are active and in place, this redundancy in the system makes it effective. Such a strategy also has direct benefits for the system's overall robustness and dependability. Then, the generic use of NeuroDots will be achieved through a combination of a) application-specific placement (i.e., the topology) of multiple, identical devices; b) their network-level synergy; and c) their application-specific programming.

Hierarchical/multiscale computing. Though homogeneous at the ‘ant-implant’ level, we are using a hierarchical (and, if the application benefits, multiscale) computing architecture, i.e., multiple open or closed loops, with varying degrees of processing power, latency, etc. within the NeuroDots. The reason for imbuing implants with wireless access to the outside world is to be able to offload data logs. Secondarily, another reason is to be able to update the operational parameters or, even, firmware of those devices in the field, that is, after implantation. However, wireless capabilities have become less esoteric—e.g., by the adoption of industry-standard protocols such as Bluetooth—and more prominent over time, allowing for larger data volumes to be transferred over a longer distance. In so doing, wireless communication is—undoubtedly—improving the quality of service of modern implants. However, here we posit that it is doing far more; it is instigating a paradigm shift for future implants: though still located inside the body, implants can now expand their functionality outside the human body. They will still interact with living tissue, yet they will also be able to tap into computational and other resources available outside and, oftentimes, far from the patients themselves. As an example, imagine a case of a future seizure-prevention network neuromodulator being located inside the human skull. Whereas previously seizures were related to one single trigger area, it has become evident that seizures are an emergent property of a ‘seizure network’, that is characterized by increased functional connectivity between different seizure nodes. Though it records neural activity from multiple sites subdurally, in order to deal with the problem of timely prevention, it may enlist the closed-loop synergy of all dot implants to localize and suppress a seizure onset within the ‘seizure network’. If the local synergy is not enough to properly identify and stop the seizure due to any number of reasons, it may also enlist the clinical statistics residing in some remote resource for proper seizure detection, subject to patient age, gender or even geographic location. This may require the added processing power and centralized overview of a master (cranial) implant coordinating and aggregating the data from all other dot nodes. That implant should be closer to the skull (e.g., like a modern NeuroPace device and should be equipped with higher compute and wireless capabilities.

Federated or collaborative learning and other distributed strategies may be facilitated by such an implant. If this slower but more powerful loop is still not sufficient or if more systemic phenomena are taking place outside the strict purview of the implant network—e.g., patient stimulation parameters have drifted, a local patient group exhibits different seizure patterns due to a change in living conditions such as conflict, pollution, traffic, regional medication policies—then implant-system functionality has to expand once more, now outside the patient and into wirelessly collaborating resources, e.g. Big Data available in a medical data lake in the Cloud. Trying to make sense of their patient's ‘local’ readings may then become much easier by comparing and contrasting against (anonymized) population-wide statistics. Through even more powerful computing capabilities in the Cloud, such comparisons can help indicate a drift in operational parameters, in patient-specific or time-sensitive stimulation strategies, and so on. Tiny implants will need to transfer (proportionally speaking) large volumes of physiological data upstream, while a powerful Cloud server will process the data and make a decision which usually translates to simple or little data traveling downstream. Adaptive & self-learning devices: The above requirement makes it obvious that the neuromodulatory dot system of the present invention system needs to detect patterns in the acquired signals. The problem of pattern recognition in neural signals is well-studied in the literature and has received renewed interest due to the emergence of deep neural networks. However, in the context of networked devices that are potentially also connected to the Cloud, the devices can cooperate, and certain computations can be delegated to the Cloud. Such a distributed setting leads to considerations of how best to utilize the ever-improving capabilities of artificial intelligence (AI). For instance, consideration of those parts of the data processing and AI modeling shall be done by individual devices, what AI computations can be done jointly by multiple devices in tandem, and what AI processing can be carried out by the cloud. If data from devices implanted in many patients is available, AI models can be trained in real time on data from multiple patients. This may lead to more accurate machine-learning models if data for a particular patient is limited. A model can be trained first on data from other patients, and then be fine-tuned on data of the target patient by means of transfer learning. To reduce the communication load and protect the privacy of the patients, train AI models locally on each device individually, and only share the parameters of the model across the different devices, instead of sharing neural data. The framework of federated learning provides algorithmic approaches for distributed learning of AI models based on data stored at multiple sites. Although federated learning is usually applied in the context of medical datasets that are safeguarded at multiple hospitals, it can also be explored in the context of multiple devices that gather neural data, where AI models are trained on data from those devices without sharing neural data between the devices.

Efficient data reduction/handling. The implantable nodes will generate a data deluge that needs to be efficiently and effectively dealt with. Ideally, one would want to reduce the data volume as much as possible, while preserving information for self-learning and closed-loop control in later steps in the process. This is precisely the objective of data compression, a well-studied topic in the fields of signal and information processing and information theory. Data compression can be classified into two major categories, lossy and lossless compression. The former discards some components of the neural signals, and therefore, compresses the signals substantially, whereas the latter allows perfect reconstruction of the neural data, and as a consequence, only modestly compresses the original data. For clinical applications, exact reconstruction of neural signals is more important than data reduction. In other applications, lossy compression may be more suitable. A compelling compromise between lossless and lossy compression is “near-lossless” compression. Attractive compression rates (e.g., 3 to 10 fold) can be achieved while the attendant distortion remains acceptable. In near-lossless compression, no sample in the reconstructed signal is changed in magnitude more than a fixed, positive tolerance level compared to the original sample. Neural signals are typically analyzed by visual inspection by human experts and/or by automated analysis using signal processing and machine learning algorithms. Therefore, compression of neural data would only be suitable as long as compression does not introduce any errors in such analysis. In this context, near-lossless compression is often adequate, as the user can control the maximum amount of distortion. For the design of compression schemes for multi-sensor neural data, there are for example the following factors.

Firstly, as suggested above, the distortion in each signal sample should be below a tolerance set by the clinician (near-lossless compression). Secondly, the compression scheme should exploit the inter- and intra-channel correlation, i.e., the correlations over time and across the different sensors (channels). Thirdly, the compression scheme should support progressive transmission, where the first transmitted bits allow a coarse reconstruction of the neural data, and gradually the quality of the reconstruction improves as more bits become available. In other words, the bitstream can be truncated at any point below the encoded rate to give the best quality reconstruction at that particular rate.

In the following, we briefly address each of these three properties. Near-lossless compression algorithms typically consist of two stages. First the multi-sensor data is subjected to lossy compression, next the residual of the lossy reconstructed data is quantized and compressed in a lossless fashion (often by arithmetic coding). The latter step makes it possible to bound the distortion on the residual for each individual sample, and hence also the reconstructed neural data. Inter- and intra-channel correlations can be exploited by arranging neural data in matrices or tensors. Once the data is arranged in this multi-dimensional manner, it can be analyzed by matrix and tensor decomposition methods or via deep learning in neural networks. An alternative framework is compressed sensing, which relies on sparcity in the (potentially multidimensional or multi-scale) representations of signals.

Progressive transmission can be achieved by representing the neural data on multiple scales, such as discrete or continuous wavelets or Gabor decompositions. By decoding the lowest scale in the representation first, followed by the higher scales, the signal reconstruction gradually improves as more data is received. Beside the algorithmic aspects, another challenge is to design compact and energy efficient hardware to support compression of neural signals where compression could be performed at the sensors directly. Moreover, instead of processing and compressing all neural data, only relevant events in the signals could be compressed and transmitted (e.g., epileptic seizures), reducing the overall computational and communications load of the neural platform.

Secure computation & communication. The data generated by the dot implants introduced in the above section is of highly sensitive and private nature. The most natural form of securing this data is using wired communication. However, in the case of wireless dot implants additional measures need to be employed to properly secure the wireless interface from eavesdropping, malicious implant access, data tampering, and so on. Unfortunately, these tiny implants have very limited computational resources to execute computationally expensive cryptographic primitives needed to secure wireless communication. This calls for employing unconventional security solutions for this purpose. For example, we discussed that ultrasound is being touted as an in-body communication channel between these implants. This channel is inherently secure when the popular MHz-range ultrasound transducers are employed. This can relieve the need for employing any cryptographic computations on these implants. However, ultrasound offers limited bandwidth and thus data rates, and is severely attenuated by the skull. As a result, additional (cranial) implants need to be employed that interface the tiny dot implants with the outside world. More specifically, for the above security solution a different communication medium than ultrasound is required when talking to an external wearable device. This also implies that these cranial implants need to be relatively larger than the dot implants in order to house the cryptographic primitives needed to secure the external wireless channel.

Dependable operation. By definition, every life-critical system such as a medical implant needs to come with sufficiently high levels of dependability and so do the NeuroDots. Here we will mostly focus on two levels of dependability for NeuroDots. At the device level, and dot implants need to be resilient and robust, ensuring chronic and correct functionality. Various techniques can be employed including hardware and software fault tolerance. At the network level, combining multiple identical dot nodes, device failure rates can be better controlled and, what is more, graceful degradation via self-organizing implants can be achieved. If one or a few devices fail, the rest can go on effecting network neuromodulation, avoiding catastrophic failure, and more preferably suffering only diminished effectiveness. This property is to be achieved by providing some redundant nodes to the NeuroDots network. The aim of the dot redundancy will be to some extent to shadow the redundancy of the biological brain.

There are now set forth examples of system architectures, or topologies.

Dot implants only. The first topology refers to the ideal case, in which mm-sized dot implants directly communicate with the outside world (i.e. in FIG. 19 the cloud 1912 via a portable device 1910, e.g. a smartphone) With regard to powering such nodes, mm-sized batteries for such implants are designed. On the other hand, continuously transferring power wirelessly from the portable device is an alternative embodiment, though a hazardously large amount of power needs to be applied in order for the required amount to effectively reach the dot implants. Secondly, for a large number of recording channels, it would become infeasible for the dot implants 1906, 1908, to directly transmit the bulky recorded data to the portable device 1910 without relentless data compression. Moreover, this compression or dimensionality reduction is not feasible to implement on these tiny nodes because it is severely limited in resources for the needed computation. In addition, for high usability, it makes sense to use the technologies that are already available on the portable device 1910, such as Bluetooth, near-field communication (NFC), etc. But these technologies and respective security standards are still too bulky to be executed on these mm-sized nodes, e.g., due to computational and security requirements, transmit powers, area requirements, etc.

Dot implants+cranial implant. In this topology, a cranial implant 1904, 1909, is added between the dot implants and the external world. This cranial implant 1904 has a battery and can wirelessly transfer power to the nodes, 1905 while acting as a communication relay 1903 between them and the outside world. Since the cranial implant 1904 is significantly larger in size compared to the dot implants 1906, 1908, it can run more complex algorithms in order to provide a local closed loop. This also reduces the amount of data that needs to be transmitted externally. Finally, compared to Topology 1, the security requirements on the nodes can be relaxed, since usually a very localized and short-range communication channel is employed to interface the neuromodulatory dots and the cranial implants. In case a non-rechargeable battery is used in the cranial implant 1904, the operational lifetime of the system is limited unless another surgery is performed to replace the battery.

Dot implants+cranial implant+wearable node. In this topology, a wearable component is added that holds the battery, whereas the cranial implant 1904 only has an energy reservoir and acts as a relay node between the dot implants and the wearable node. The main advantage of this configuration is that it will allow for reduced invasiveness, for improved physical access and thus for improved maintainability and easier (re)charging or battery replacement. However, the patient is now required to wear the wearable device, which impacts usability. The rest of the considerations are the same as for the topology of Dot Implants+Cranial Implants. Some examples of the implantable portion of this topology include the Neural Dust and the Neurograin approach. However, it should be noted that their focus is only on implant miniaturization, wireless power transfer and data communication between the different tiers.

Dot Implants+Wearable Node. This topology comprises dot implants and the wearable device and it avoids the implantation of a cranial node. However, such a scenario has its own engineering challenges since it is difficult to transfer power and communicate directly with the deeply implanted dot nodes. Ultrasound-based power transfer may be feasible but is challenged if the skull (which is opaque to ultrasound) is present between the dot implants and the wearable node. The lack of feasibility of ultrasound impacts the available communication options as well, i.e., it cannot be used as a communication channel. Consequently, security also becomes a concern since the employed communication channel (i.e., other than ultrasound) will be vulnerable to eavesdropping. This is because alternative communication mediums, such as RF, are not inherently secure the way that ultrasound is secure. This implies that the dot implants need to encrypt the data in order to secure the communication channel with the wearable device, which complicates the design of these small devices.

Threads. There are no dot implants in this topology. However, there are deeply implanted electrodes that are connected with a cranial implant via thin threads. The advantage of this topology is that there is no processing power required near the electrodes and thus all the computations can be localized at the cranial implant. Moreover, the wired connection between the electrodes and the cranial implant does away with the wireless transceivers and the associated challenges. Other characteristics of this topology include the fact that data compression is not required at the electrode level since it is not being transmitted wirelessly. The wired connection also implies that the data transmitted via the threads is inherently secure. The downside of this topology is that the implantation of these wires originating from one location to different locations spread over the brain is unresolved. We term this as the spaghetti problem. Moreover, similar to the topology of Dot Implants+Cranial Implant, surgery is required to replace the cranial implant in case of a non-rechargeable battery. Example systems of this type of topology include Neuropace.

Threads+Wearable Node. This topology has a wearable node that communicates with the cranial implant. As a result, the cranial-implant battery can be moved to the wearable node which resolves the battery replacement issue of the previous topology. However, now the patient needs to continuously wear this wearable device, which impacts usability. Example systems of this type of topology include Neuralink.

As contemplated in embodiments herein, a predetermined stimulation site for tissue of interest can include either peripheral neuronal tissue and/or central neuronal tissue. Neuronal tissue includes any tissue associated with the peripheral nervous system or the central nervous system. Peripheral neuronal tissue can include a nerve root or root ganglion or any neuronal tissue that lies outside the brain, brainstem or spinal cord. Peripheral nerves can include, but are not limited to olfactory nerve, optic, nerve, oculomotor nerve, trochlear nerve, trigeminal nerve, abducens nerve, facial nerve, vestibulocochlear (auditory) nerve, glossopharyngeal nerve, vagal nerve, accessory nerve, hypoglossal nerve, suboccipital nerve, the greater occipital nerve, the lesser occipital nerve, the greater auricular nerve, the lesser auricular nerve, the phrenic nerve, brachial plexus, radial axillary nerves, musculocutaneous nerves, radial nerves, ulnar nerves, median nerves, intercostal nerves, lumbosacral plexus, sciatic nerves, common peroneal nerve, tibial nerves, sural nerves, femoral nerves, gluteal nerves, thoracic spinal nerves, obturator nerves, digital nerves, pudendal nerves, plantar nerves, saphenous nerves, ilioinguinal nerves, gentofemoral nerves, and iliohypogastric nerves.

Central neuronal tissue includes brain tissue, spinal tissue or brainstem tissue. Brain tissue can include thalamus/sub-thalamus, basal ganglia, hippocampus, amygdala, hypothalamus, mammilary bodies, substantia nigra or cortex or white matter tracts afferent to or efferent from the abovementioned brain tissue, inclusive of the corpus callosum. Spinal tissue can include the ascending and descending tracts of the spinal cord, more specifically, the ascending tracts of that comprise intralaminar neurons or the dorsal column. The brainstem tissue can include the medulla oblongata, pons or mesencephalon, more particular the posterior pons or posterior mesencephalon, Lushka's foramen, and ventrolateral part of the medulla oblongata.

A doctor, the patient, or another user of an implantable stimulator may directly or indirectly input or program designed therapy parameters to specify or modify the nature of the stimulation provided.

An IMD may include an implantable wireless receiver. An example of a wireless receiver that may be commercially obtained and used in this embodiment of the present invention is one that is manufactured by Advanced Neuromodulation Systems, Inc., such as their Renew® System, part numbers 3408 and 3416. In another embodiment, an IMD can be optimized for high frequency operation as described in U.S. Pat. No. 7,450,987, entitled “Systems And Methods For Use In Pulse Generation,” the entire written and illustrative disclosure of which is incorporated herein by reference. The wireless receiver is capable of receiving wireless signals from a wireless transmitter that is located external to the patient's body. A doctor, the patient, or another user of the IMD may use a controller located external to the person's body to provide control signals for the operation of the IMD. A controller thus provides control signals to a wireless transmitter, which in turn transmits control signals and power to the wireless receiver of the IMD. Having received the designed control signals, the IMD uses the control signals to vary the signal parameters of electrical signals that are transmitted through one or more electrical stimulation leads to one or more stimulation sites, preferably in the brain. The external controller can, in one alternative embodiment comprise a handheld programmer, to provide a means for programming the IMD. An example of a wireless transmitter that may be obtained commercially and that are manufactured by Advanced Neuromodulation Systems, Inc., are such as the Renew® System, their part numbers 3508 and 3516.

The selected IMD can, for example be programmed by the therapy designer to apply any desired stimulation wave or combination of stimulation waves, including for example infraslow or slow stimulation waves or nested stimulation, particularly where the electrical pulses are in the waveform of a chosen color, for example pink noise, grey noise, or blue noise, that is nested upon an electrical pulse in the waveform of an infraslow or slow carrier wave, or it can for example alternatively by programmed to apply a burst-type electrical pulse stimulation to targeted brain tissue of a patient, or to apply burst stimulation sequentially combined with noise stimulation in any of the noise stimulation waveforms that may be used in therapy. Specifically, the IMD includes a microprocessor and a pulse generation module. The pulse generation module generates the electrical pulses according to a defined pulse width and pulse amplitude and applies the electrical pulses to defined electrodes. The microprocessor controls the operations of the pulse generation module according to software instructions stored in the device.

An IMD can be adapted by programming its internal microprocessor to deliver a number of spikes (relatively short pulse width pulses) that are separated by an appropriate interspike interval. Thereafter, the programming of the microprocessor causes the pulse generation module to cease pulse generation operations for an interburst interval. The programming of the microprocessor also causes a repetition of the spike generation and cessation of operations for a predetermined number of times. After the predetermined number of repetitions has been completed within a nested stimulation waveform, the programmed microprocessor can cause burst stimulation to cease for an amount of time (and resume thereafter). Also, in some embodiments, the microprocessor could be programmed to cause the pulse generation module to deliver a hyperpolarizing pulse before the first spike of each group of multiple spikes.

The microprocessor can be programmed to allow the various characteristics of the burst stimulus to be set by a physician to allow the burst stimulus to be optimized for a particular pathology of a patient. For example, the spike amplitude, the interspike interval, the interburst interval, the number of bursts to be repeated in succession, the electrode combinations, the firing delay between nested stimulation waveforms delivered to different electrode combinations, the amplitude of the hyperpolarizing pulse, and other such characteristics could be controlled using respective parameters accessed by the microprocessor during burst stimulus operations. These parameters could be set to desired values by an external programming device via wireless communication with the implantable neuromodulation device.

In alternative representative embodiments, an IMD may apply electrical stimulation according to a suitable noise signal by which is meant white noise, pink noise, purple noise, blue noise, grey noise, brown noise, black noise, or green noise. Details regarding implementation of a suitable noise signal can be found in U.S. Pat. No. 8,682,441, the entire disclosure of which is incorporated herein by reference. In another embodiment, an IMD may be implemented to apply burst stimulation using a digital signal processor and one or several digital-to-analog converters. The burst stimulus waveform could be defined in memory and applied to the digital-to-analog converter(s) for application through electrodes of the medical lead. The digital signal processor could scale the various portions of the waveform in amplitude and within the time domain (e.g., for the various intervals) according to the various burst parameters.

A nested stimulation (NS) system may be controlled to deliver various types of nested stimulation therapy, such as high frequency neurostimulation therapies, burst neurostimulation therapies and the like. High frequency neurostimulation includes a continuous series of monophasic or biphasic pulses that are delivered at a predetermined frequency, most preferably color noise stimulation waveform frequency patterns nested upon a low frequency waveform and designed so as to produce a reinstated normal anticorrelation connectivity communication between for example the brain's salience plus central executive network and the brain's default node network. The low frequency waveforms include infraslow waveforms with pulse widths of 1000 seconds to 10 seconds, which is called infraslow stimulation, or 10 seconds to 1 second, which is called slow stimulation. These infraslow and slow waveforms can be programmed to be anticorrelated, correlated or uncorrelated within or between different brain networks. Burst neurostimulation can be nested on top of the noise waveforms or as sole nested waveform without the noise. Bursts include short sequences of monophasic or biphasic pulses, where each sequence is separated by a quiescent period.

The NS system may deliver nested stimulation therapy based on preprogrammed therapy parameters. The therapy parameters may include, among other things, pulse amplitude, pulse polarity, pulse width, pulse frequency, interpulse interval, inter-burst interval, electrode combinations, firing delay and the like. Optionally, the NS system may represent a closed loop neurostimulation device that is configured to provide real-time sensing functions from a lead. The configuration of the lead sensing electrodes may be varied depending on the neuronal anatomy of the sensing site(s) of interest. The size and shape of electrodes is varied based on the implant location. The electronic components within an NS system may be designed with both stimulation and sensing capabilities, including alternative nested stimulation therapy, such as burst mode, high frequency mode and the like.

An NS system may include an implantable medical device (IMD) that is adapted to generate designed electrical pulses for application to tissue of a patient. The IMD typically comprises a metallic housing or can that encloses a controller, a pulse generating circuit, a charge storage circuit, a battery, a far-field and/or near field communication circuit, a battery charging circuit, a switching circuit, a memory circuit, and the like. A suitable charge storage circuit may represent one or more capacitors and/or battery cells that store charge used to produce the therapies described herein. The pulse generating circuitry, under control of the controller, manages discharge of the charge storage circuit in order to shape the morphology of the waveform delivered while discharging energy. The switching circuitry thus connects select combinations of the device's electrodes a pulse generating circuitry, thereby directing the stimulation waveform to a desired electrode combination. As explained herein, such switching circuitry successively connects the pulse generating circuitry to any variety of successive electrode combinations. A controller typically includes one or more processors, such as a microcontroller, for controlling the various other components of the device. Software code is typically stored in memory of an IMD for execution by the microcontroller or processor to control the various components of the device. An IMD may comprise a separate or an attached extension component. If the extension component is a separate, discrete component, the extension component may connect with the “header” portion of the IMD, as it is known in the art. If the extension component is integrated with the IMD, internal electrical connections may be made through respective conductive components. Within the IMD, electrical pulses are generated by a suitable pulse generating circuit and are provided to a switching circuitry. A switching circuitry connects to outputs of the IMD. Electrical connectors (e.g., “Bal-Seal” connectors) within the connector portion of an extension component, or within the IMD header may be employed to conduct various stimulation pulses. The terminals of one or more leads are inserted within a connector portion or within the IMD header for electrical connection with respective connectors. In this fashion, the pulses originating from the IMD are provided to the lead. Any pulses generated are then conducted through the conductors of a lead and applied to selected tissue of a patient via conventional stimulation electrodes that are coupled to blocking capacitors. Any suitable known or later developed design may be employed for a connector portion.

Stimulation electrodes may be positioned along a horizontal axis of a lead, and may be, for example, angularly positioned about the horizontal axis, so that the stimulation electrodes aren't arrayed so as to overlap. Adjacent stimulation electrodes may be separated from one another by non-conducting rings that may electrically isolate each stimulation electrode from any adjacent stimulation electrode. Such non-conducting rings may include one or more insulative materials and/or biocompatible materials to allow a lead to be implantable within the patient. Non-limiting examples of such materials include polyimide, polyetheretherketone (PEEK), graphene, polyethylene terephthalate (PET) film (also known as polyester or Mylar), polytetrafluoroethylene (PTFE) (e.g., Teflon), or parylene coating, polyether bloc amides, polyurethane. Stimulation electrodes may be configured to emit their pulses in an outward radial direction proximate to or within a stimulation target. The stimulation electrodes may deliver noise, tonic, high frequency and/or burst nested stimulation waveforms as described herein. Optionally, the electrodes may usefully also sense neural oscillations and/or sensory action potential (neural oscillation signals) for a data collection, identification and/or recordation window in near-simultaneous time, enabling a suitably programmed IMD to generate waveforms as desired by the clinician to be applied to the patient in response to the sense oscillations detected and identified.

A lead may comprise a lead body of insulative material about a plurality of conductors within the material that extend from a proximal end of the lead, proximate to the IMD, to its distal end. Conductors electrically couple a plurality of the stimulation electrodes to a plurality of terminals of the lead. Terminals are adapted to receive electrical pulses, and stimulation electrodes may be adapted to apply generated pulses to the stimulation target of the patient. Also, sensing of physiological signals may occur through stimulation electrodes, conductors, and terminals. Although not required for any particular embodiments, the lead body of a lead may be fabricated to flex and elongate upon implantation or advancing within the tissue (e.g., nervous tissue) of the patient towards the stimulation target and for accomodating movements of the patient during or after implantation.

By way of example, an IMD may include a processor and associated charge control circuitry as described in U.S. Pat. No. 7,571,007, entitled “Systems and Methods For Use In Pulse Generation”, the entire written and illustrative disclosure of which is incorporated herein by reference. Circuitry for recharging a rechargeable battery (e.g., suitable battery charging circuitry) of an IMD using inductive coupling and external charging circuits are described in U.S. Pat. No. 7,212,110, entitled “Implantable Device And System For Wireless Communication”, the entire written and illustrative disclosure of which is incorporated herein by reference. An example and discussion of “constant current” pulse generating circuitry is provided in U.S. Patent Publication No. 2006/0170486A1 entitled “Pulse Generator Having An Efficient Fractional Voltage Converter And Method Of Use”, the entire written and illustrative disclosure of which is incorporated herein by reference.

Different burst and/or noise high frequency pulses nested on low frequency pulses on different stimulation electrodes may be generated using a single set of pulse generating circuitry using consecutively generated pulses according to a “multi-stimset program” as they are known in the art, or by using multiple sets of pulse generating circuitry. Complex pulse parameters may be employed such as those described in U.S. Pat. No. 7,228,179, entitled “Method And Apparatus For Providing Complex Tissue Stimulation Patterns”, and International Patent Publication Number WO2001/093953 A1, entitled “Neuromodulation Therapy System,” the entire written and illustrative disclosures of each of them being incorporated herein by reference. Alternatively, multiple sets of such circuitry may be employed to provide such varied pulse patterns. Various sets of parameters may define the pulse characteristics and pulse timing for the pulses applied to various stimulation electrodes. Although constant current pulse generating circuitry is contemplated for some embodiments, any other suitable type of pulse generating circuitry may be employed such as constant voltage pulse generating circuitry.

A controller may deliver a nested stimulation waveform to at least one electrode combination located proximate to nervous tissue of interest, the nested stimulation waveform including a series of pulses configured to excite, for example nested C-fibers of nervous tissue of interest, the nested stimulation waveform and the carrier waveform defined by therapy parameters or preprogrammed therapy parameters that are based upon information collected from numerous past patients, patient clinical trials, medical literature, and/or tests performed upon an individual patient during initial implant and/or during periodic checkups.

In simultaneous signal recordation and stimulation, a controller can sense intrinsic neural oscillations from at least one of the electrodes on a lead. The controller may analyze such intrinsic neural oscillations signals to obtain useable brain activity data. Then, the controller can determine whether the collected (and recorded) activity data satisfies some selected criteria of interest i.e. normal or baseline functionality. The controller then adjusts at least one of the therapy parameters to change the carrier and nested stimulation waveform or waveforms selected, when the activity data does not satisfy the criteria of interest. The controller then iteratively repeats such delivering operations for a group of TPS. The IMD selects a candidate TPS from the group of TPS known to be based on a criteria of interest. The controller may repeat the delivering, sensing and adjusting operations to optimize the nested stimulation waveform. The analyzing operation may include analyzing a feature of interest from a morphology of the neural oscillation signal over time, counting a number of occurrences of the feature of interest that occur within the signal over a predetermined duration, and the generation of activity data based on the number of occurrences of the feature of interest.

Memory means stores software configured to control operation of a controller for nested stimulation therapy as explained herein. The memory may also stores neural oscillation signals, therapy parameters, neural oscillation activity level data, sensation scales and the like. For example, the IMD's memory may save neural oscillation activity level data for various different therapies as applied over a short or extended period of time. A collection of neural oscillation activity level data is accumulated for different therapies and may be compared to identify high, low and acceptable amounts of sensory activity.

A controller device may be implemented to manage the processes of the charge and recharge a suitable battery of an IMD (although a separate recharging device could alternatively be employed) and to program an IMD on the pulse specifications while implanted within the patient. In alternative embodiments, separate programmer devices may be employed for charging, recharging and/or programming the IMD. The controller device selected may be a processor-based system that possesses wireless communication capabilities. Software may be stored within a non-transitory memory of the controller device which may be executed by the processor to control the various operations of the controller device. An electronic wand-like device may be electrically connected to the controller device through suitable electrical connectors. Electrical connectors may be electrically connected to a telemetry component means (e.g., an inductor coil, or an RF transceiver) at the distal end of such a wand through respective wires allowing bi-directional communication with the IMD. Optionally, in alternative embodiments, the wand-like device may comprise one or more temperature sensors for use during charging operations.

The user may initiate communication with a preferred embodiment of the IMD by placing the wand proximate to the NS system. Preferably, the placement of the wand will allow a telemetry system within the wand to be aligned with far-field and/or nearfield communication circuitry installed in an IMD. A controller device will preferably provides one or more user interfaces (e.g., a touchscreen, a keyboard, a mouse, one or more buttons, or the like) allowing the user to operate the IMD. The controller device may be controlled by the user (e.g., physician, nurse, therapist) through the user interface, thereby allowing the user to interact with the IMD. The user interface of choice may permit the user to move electrical stimulation along and/or across one or more lead(s) using different stimulation electrode combinations, for example, as described in U.S. Patent Application Publication No. 2009/0326608A1, entitled “Method Of Electrically Stimulating Tissue Of A Patient By Shifting A Locus Of Stimulation And System Employing The Same,” the entire written and illustrative disclosure of which is incorporated herein by reference.

Alternatively, a controller device may permit operation of an IMD according to one or more therapies to treat the patient. Each therapy may include one or more sets of stimulation parameters of the pulse including pulse amplitude, pulse width, pulse frequency or inter-pulse period, firing delay, pulse repetition parameter (e.g., number of times for a given pulse to be repeated for respective stimset during execution of program), biphasic pulses, monophasic pulses, etc. In this fashion, an IIMD modifies its internal parameters in response to the control signals it receives from its controller device to vary the stimulation characteristics of stimulation pulses transmitted through a lead to the targeted tissue(s) of the patient. NS systems, stimulation headsets (stimsets), and different stimset programs are discussed in PCT Publication No. WO2001/093953A1, entitled “Neuromodulation Therapy System,” and U.S. Pat. No. 7,228,179, entitled “Method And Apparatus For Providing Complex Tissue Stimulation Patterns,” which are expressly incorporated herein by reference.

For percutaneous use, a percutaneous stimulation lead can include one or more circumferential-shaped electrodes spaced apart from one another along the length of a stimulating portion of a stimulation lead. Circumferential electrodes emit electrical stimulation energy in a substantially radial emision pattern, that is generally perpendicular to the axis of a stimulation lead, in all directions. A laminotomy, paddle, or surgical stimulation lead can include one or more directional stimulation electrodes spaced apart from one another along one surface of a stimulation lead. Although various types of stimulation leads are described herein as examples, a given embodiment of a stimulation system may include any suitable type of stimulation lead in any suitable number, and stimulation leads may be used alone or in combination. For example, medial or unilateral stimulation of the predetermined site may be accomplished using a single electrical stimulation lead implanted in communication with the predetermined site of interest in one side of the head, while bilateral electrical stimulation of the predetermined site may be accomplished using two stimulation leads implanted in communication with the predetermined site in opposite sides of the head.

In one preferred embodiment used in the practice of the invention, a stimulation source is transcutaneously in communication with a partner electrical stimulation lead. In “transcutaneous” electrical stimulation (TES), the stimulation source is external to the patient's body, and may be worn in an appropriate fanny pack or belt, and the electrical stimulation lead is in communication with the stimulation source, either remotely or directly. In another embodiment, the stimulation is percutaneous. In “percutaneous” electrical nerve stimulation (PENS), needles are inserted to an appropriate depth around or immediately adjacent to a predetermined stimulation site, and then stimulated.

An implantable stimulator can allow each electrode of each lead to be defined as having a positive, a negative, or a neutral polarity. For each electrode combination (e.g., the defined polarity of at least two electrodes having at least one cathode and at least one anode), an electrical signal can have at least a definable amplitude (e.g., voltage), pulse width, and frequency, where these variables may be independently adjusted to finely select the sensory transmitting brain tissue required to inhibit transmission of neuronal signals. Generally, amplitudes, pulse widths, and frequencies are determinable by the capabilities of the neurostimulation systems, which are known by those of skill in the art. Voltages that may be used can include, for example about 0.5 to about 10 volts, more preferably about 1 to about 10 volts. For constant current devices amplitudes may be between 0.01 and 5 m Amperes.

In different embodiments herein, the therapy parameter of signal frequency may be varied to achieve a burst type rhythm, or burst mode stimulation. Generally, the burst stimulus frequency may be in the range of about 0.01 Hz to about 100 Hz, more particularly, in the range of about 1 Hz to about 12 Hz, and still more particularly, in the range of about 1 Hz to about 4 Hz, 4 Hz to about 7 Hz or about 8 Hz to about 12 Hz for each burst. Each burst stimulus comprises at least two spikes, for example, each burst stimulus can comprise about 2 to about 100 spikes, more particularly, about 2 to about 10 spikes. Each spike can comprise a frequency in the range of about 50 Hz to about 1000 Hz, more particularly, in the range of about 200 Hz to about 500 Hz. The frequency for each spike within a burst can be variable, thus it is not necessary for each spike to contain similar frequencies, e.g., the frequencies can vary in each spike. The inter-spike interval can be also vary, for example, the inter-spike interval, can be about 0.1 milliseconds to about 100 milliseconds or any range there between. A burst stimulus is followed by an inter-burst interval, during which substantially no stimulus is applied. The inter-burst interval may have a duration in the range of about 1 milliseconds to about 5 seconds, more preferably, about 10 milliseconds to about 300 milliseconds. A burst stimulus may be configured to have a duration in the range of: about 1 millisecond to about 5 seconds; in the range of about 250 msec to 1000 msec (1-4 Hz burst firing); in the range of 145 msec to about 250 msec (4-7 Hz); in the range of 145 msec to about 80 msec (8-12 Hz); or in the range of 1 to 5 seconds in plateau potential firing. The burst stimulus and the inter-burst interval can have a regular pattern or an irregular pattern (e.g., random or irregular harmonics). More specifically, the burst stimulus can have a physiological pattern or a pathological pattern. Additional details regarding burst stimulation and stimulus patterns may be found in U.S. Pat. No. 8,897,870, the entire written and illustrative disclosure of which is incorporated herein by reference.

It is envisaged that the patient will require intermittent assessment with regard to successful or beneficial therapeutic patterns of stimulation. Different electrodes on the lead can be selected by suitable computer programming, such as that described in U.S. Pat. No. 5,938,690, the entire disclosure of which is incorporated herein by reference. The use of such a program may allows for an optimal stimulation pattern to be obtained at minimal voltages. This additionally ensures a longer battery life for implanted systems. Example fabrication processes are disclosed in U.S. Pat. No. 9,054,436, entitled, “Method Of Fabricating Stimulation Lead For Applying Electrical Stimulation To Tissue Of A Patient,” the entire written and illustrative disclosure of which is incorporated herein by reference.

Nesting on Infraslow Electrical Signals. The present invention has found a novel application of the technology and methodology for using one or more central nervous system signal noises that are nested on infraslow or slow electrical, magnetic, sound or optic signals, in order to normalize selected target communications and hence connectivity, within and in between disrupted brain networks, in order to treat central nervous system conditions and/or disorders. In an alternative embodiment, the present invention relates to a neurostimulation method of using an implantable medical device (IMD) with a pulse generator, the implantable method of which can be utilized to treat neurological, psychiatric, or psychological, conditions and/or disorders.

Neurostimulation (NS) systems are devices that generate electrical pulses and deliver the pulses to nervous tissue to treat a variety of disorders. The category of NS devices preferentially used in preferred embodiments of the present invention comprise one or more electric pulse generators. An example of their use is deep brain stimulation, which has been used to treat movement disorders such as pain, Parkinson's disease and affective disorders such as depression Recently, new stimulation configurations such as burst stimulation and high frequency stimulation, have been developed, in which closely spaced high frequency pulses are delivered. In general, conventional neurostimulation systems seek to manage pain and other pathologic or physiologic disorders through stimulation of select nerve fibers that carry pain related signals. However, nerve fibers and brain tissue carry other types of signals, not simply pain related signals. Although some neurological disorders have been treated through known neurostimulation methods, many other neurological disorders exhibit physiological complexity, functional complexity, or other complexity and have not been adequately treated through known neurostimulation methods. Consequently, no novel method of neurostimulation can be predicted to be safe and efficacious without carefully designed preclinical and clinical studies to observe the results. Hence novel neuromodulatory therapeutics cannot genuinely be found to be obvious to a practitioner of ordinary skill in this art without such testing and observation.

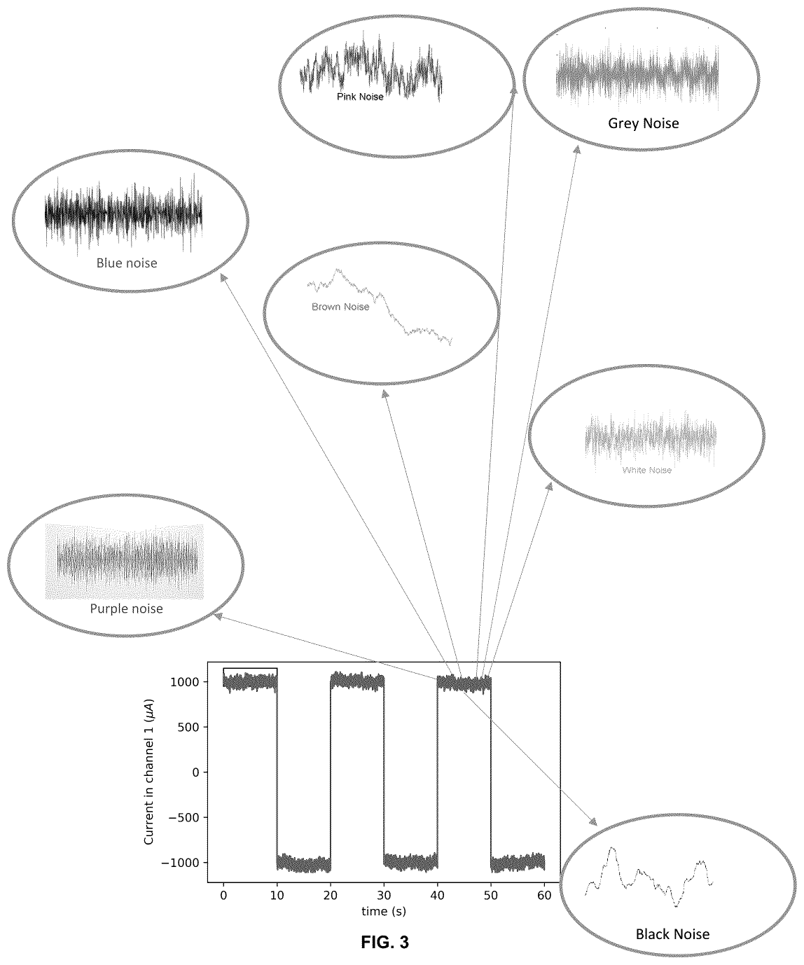

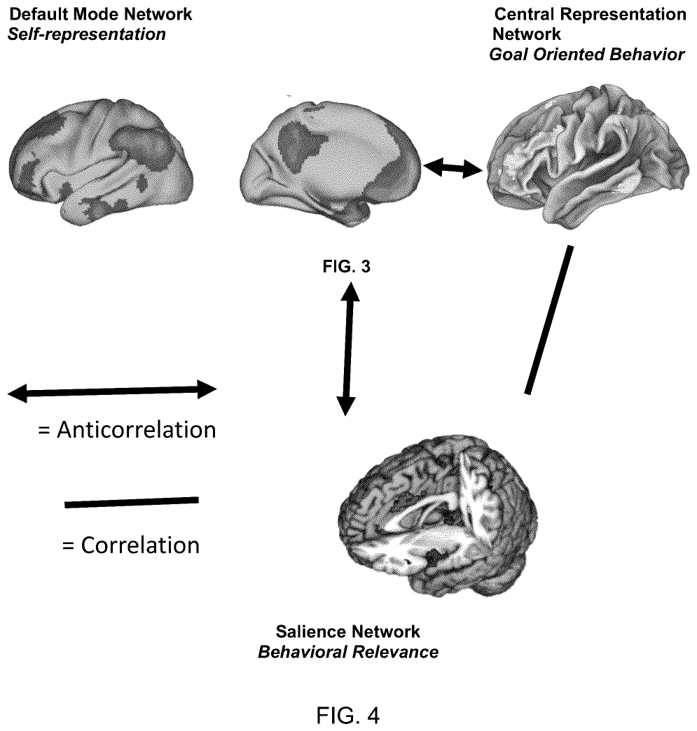

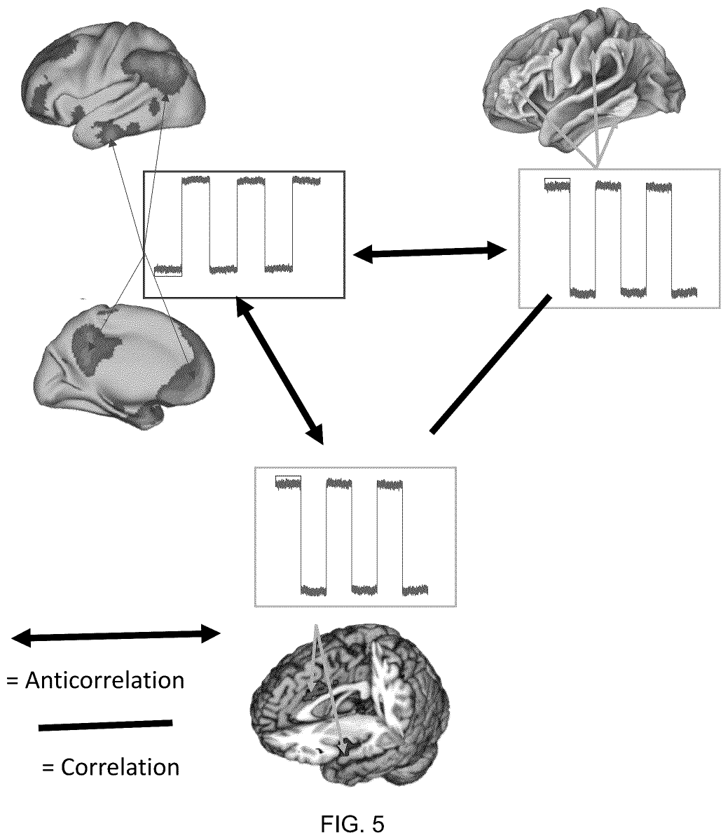

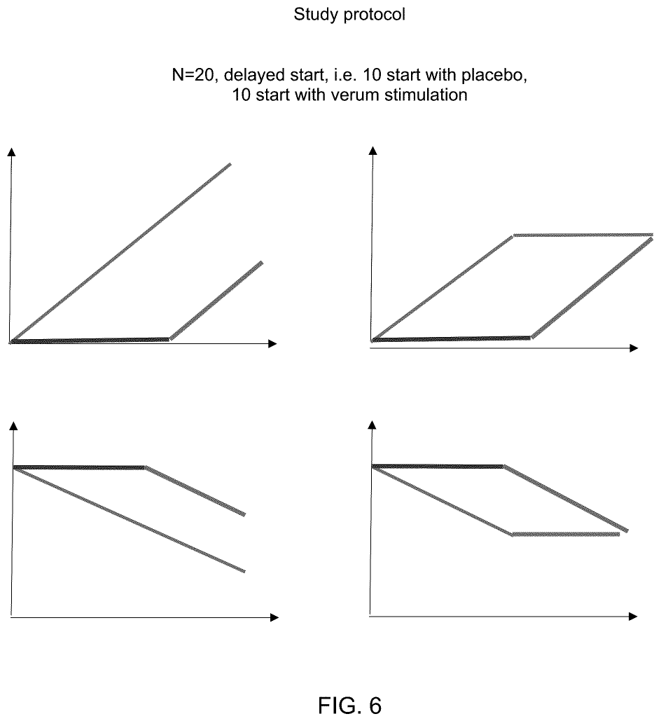

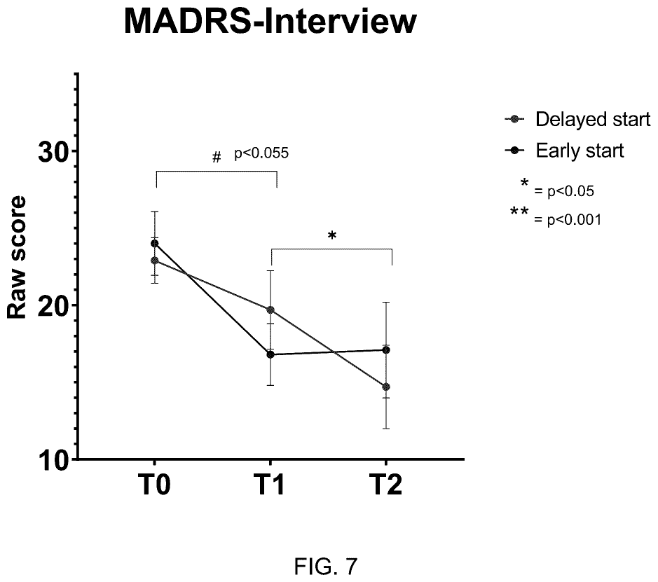

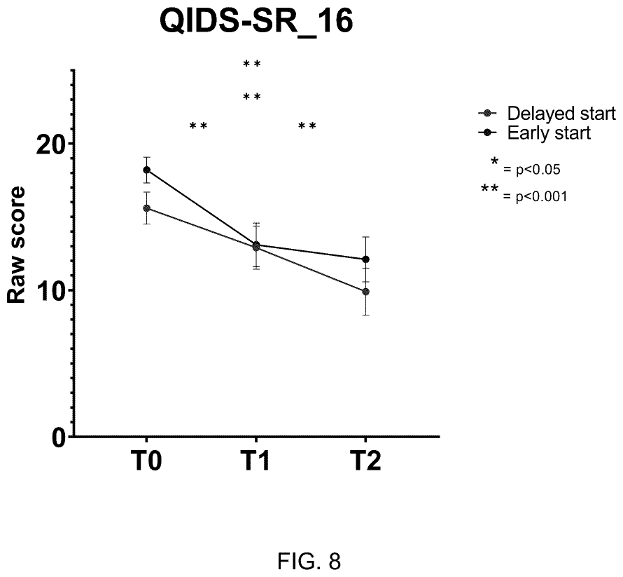

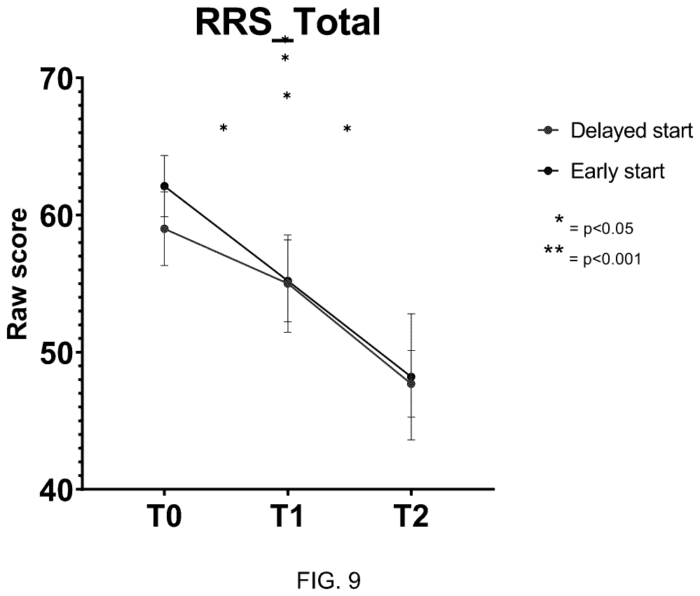

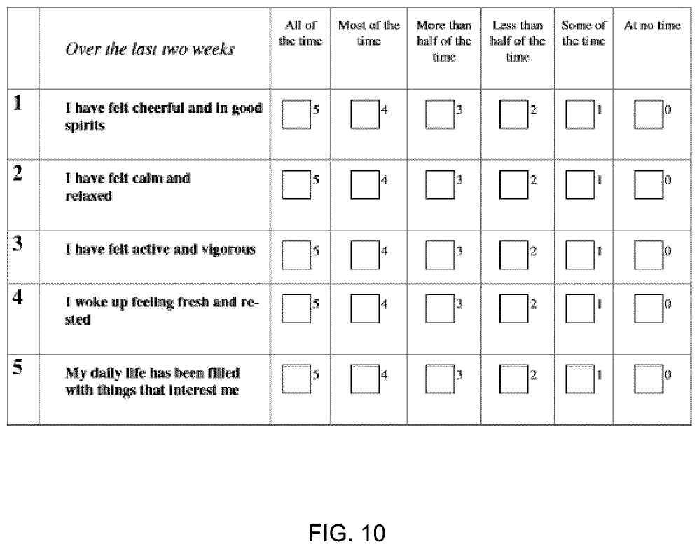

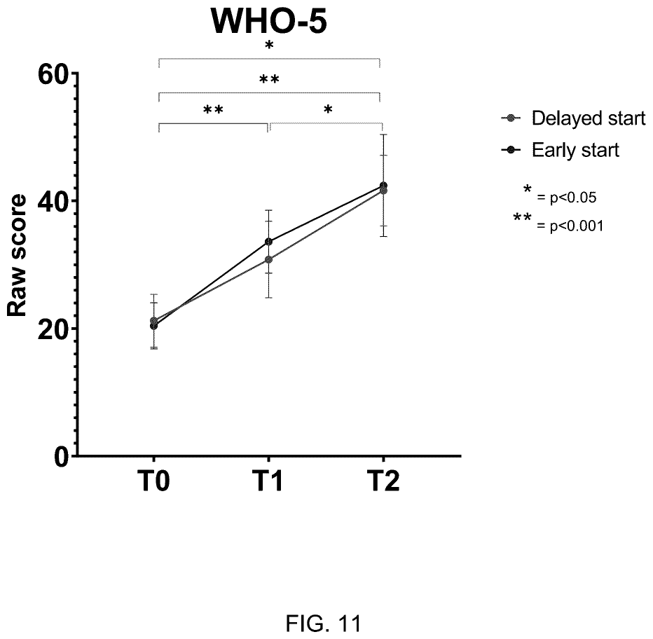

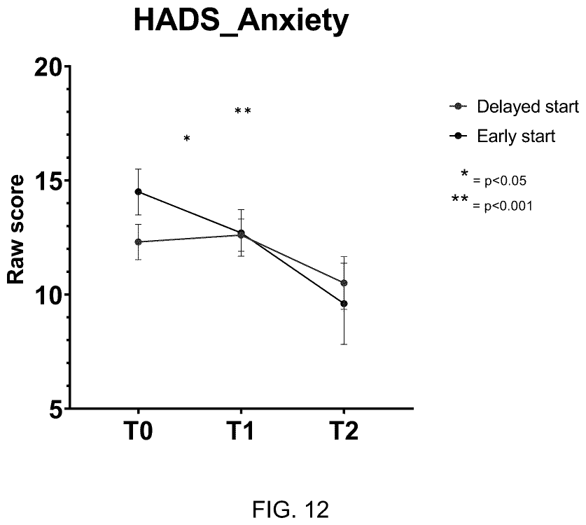

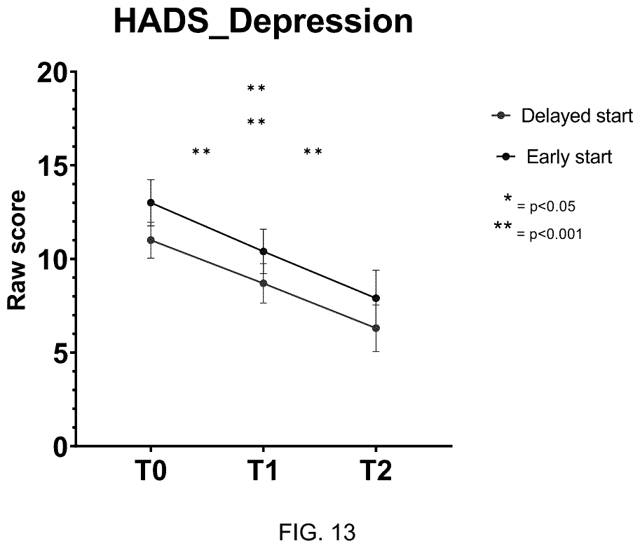

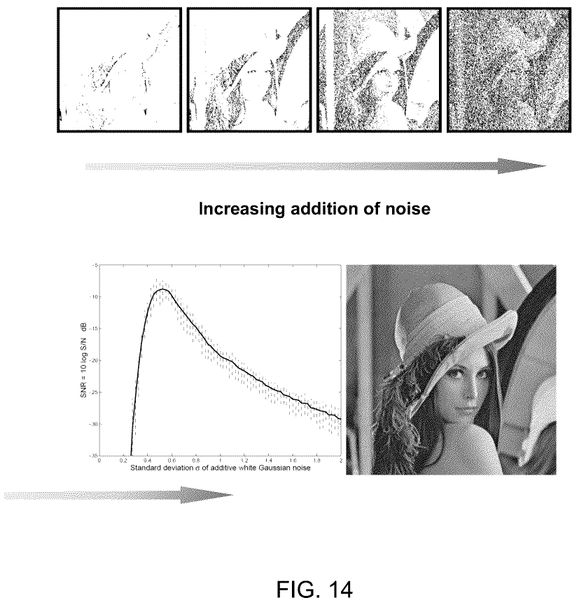

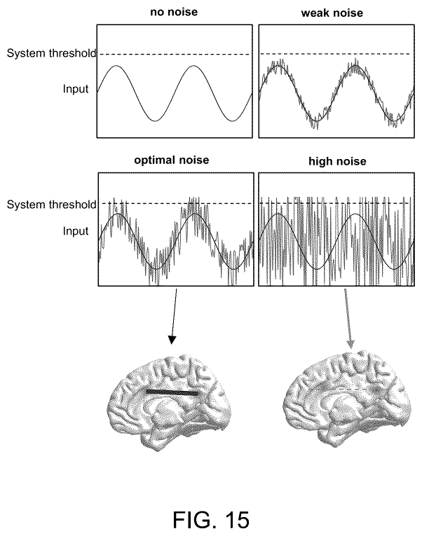

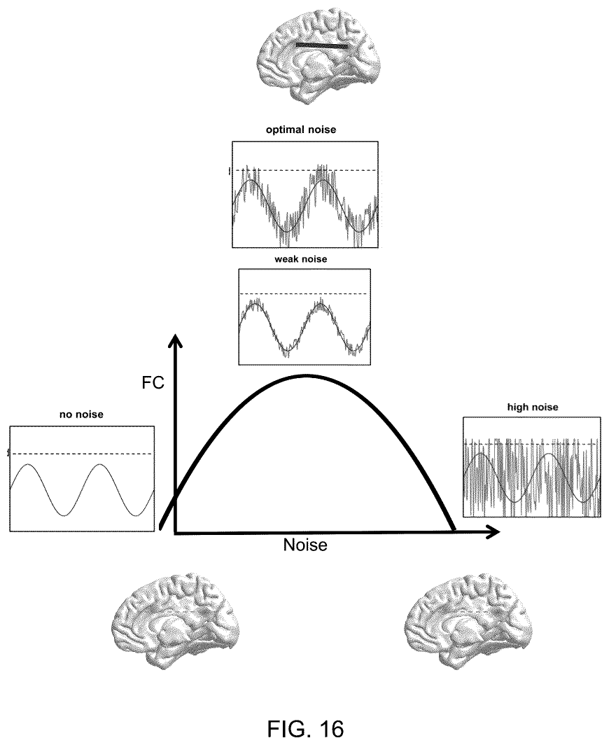

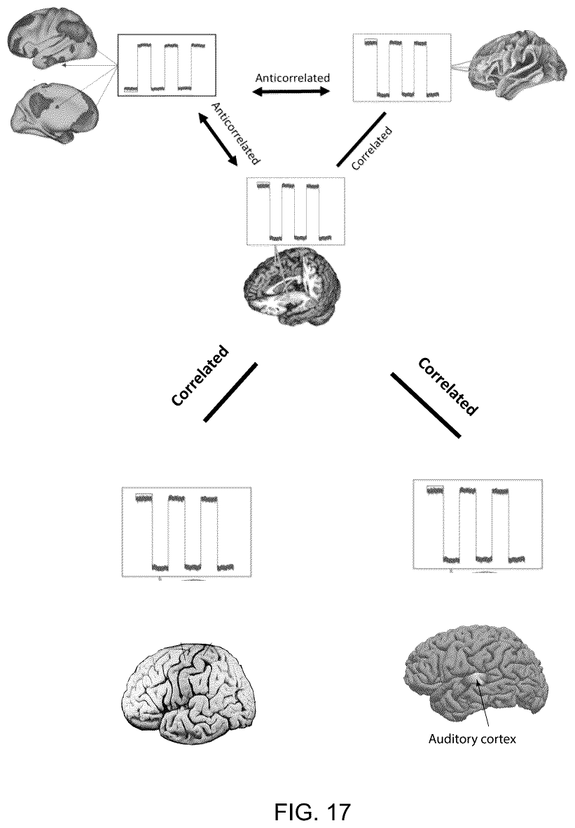

Turning to FIG. 3, there is shown a drawing of oscilloscopic waveforms that illustrate the relationship of relatively high frequency waves that are nested upon an infraslow carrier wave shown here as a function of electrical current versus time. At the upper end of one waveform burst, here in the range of approximately 1,000 microAmperes (μA), seven different classes of signal noise (colors) are shown as alternative colors that may be selected as the stimulation of choice, being nested upon the carrier wave. Specifically, there are shown in FIG. 3 the noise colors Blue, Purple, Grey, Black, Pink, Brown, Black, and White. It is to be understood that although an exemplary wave form for Green Noise is not illustrated here, that Green Noise is another noise that may be selected in the stimulation design.

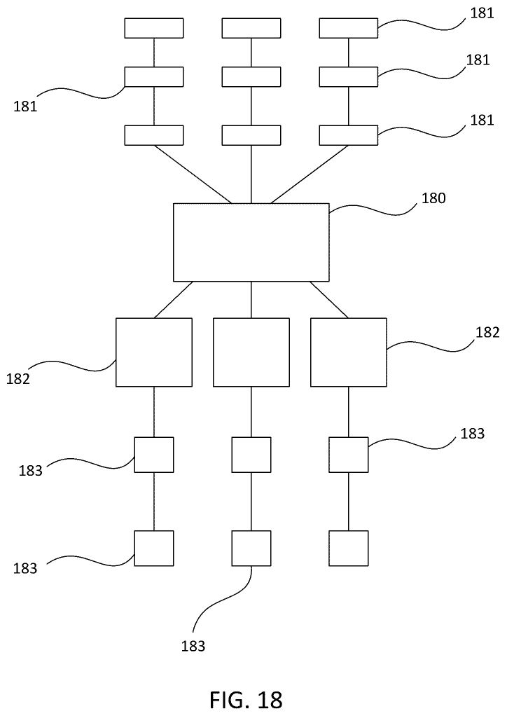

There is shown in FIG. 18 1 is a schematic drawing showing one example of a system in which a controller 180 receives sensor information from a number of sensors 181. The controller controls a number of stimulation wave generators 182, which provide stimulation waves to a number of delivery devices (e.g. electrodes) 183. The skilled reader will understand that alternative system architectures or structures are possible.

The Applicant's system therefore aims to ameliorate abnormal behaviour in brain networks by applying infraslow or slow activity to multiple brain networks. This may be done in such a way that infraslow or slow stimulation is correlated between two or more networks, and/or anti-correlated between two or more networks. For example, infraslow stimulation may be correlated between areas of the salience and central executive networks, and anticorrelated between those networks and the default mode network. In this example, normal correlated activity between the salience and central executive networks may be promoted, strengthened or restored, and normal anticorrelated activity between those networks and the default mode network may be promoted, strengthened or restored. Abnormal or pathological activity may be reduced, disrupted or ameliorated.

Noise signals, such as pink, brown, blue, purple or grey noise, or any suitable combination thereof, may be superimposed on the infraslow stimulation wave. Superimposed noise signals are nested, meaning that they are cross-frequency coupled, which can include coupling of power to power (or current density to current density), frequency to frequency, phase to phase, power to frequency, frequency to power, phase to frequency, frequency to phase, power to phase, or phase to powers. It should be understood that a superimposed signal is intended herein to also be burst or tonic, that is, not noisy via cross-frequency coupling, (i.e. phase-phase, amplitude-phase, power-power, current density-current density, phase-power, or phase current density).

Applicant's methods may be used for treating any suitable condition that involves an abnormal interaction between brain regions. This may include any suitable neurological, psychological or psychiatric condition. For example, Applicant's methods may be used in treatment of: tinnitus, epilepsy, depression, anxiety, Parkinson's Disease, autonomic dysfunctions, including cardiac, respiratory, urogenital, or gastrointestinal disorders), immune disorders, stress, attention deficit hyperactivity disorder, bipolar disorder, autism, obsessive compulsive disorder, post-traumatic stress disorder syndrome, or schizophrenia, as well as mild cognitive impairment, Alzheimers dementia Lewy-body disease dementia, multi-infarct dementia, thalamocortical dysrhythmias, tinnitus, pain, Parkinsons Disease, epilepsy, disorders of consciousness, minimally cognitive state, vegetative state, or unresponsive wakefulness syndrome.

### Alternative Preferred Embodiments

In an alternative preferred embodiment of the inventions, there is provided a method of treating a central nervous system disorder in a patient in need thereof, comprising the steps of determining the presence of an abnormal correlation interaction or an abnormal anticorrelation interaction or an abnormal uncorrelated interaction between at least one or more nodes within at least one or more identified brain networks of interest and determining whether to reinstate a desired normal anticorrelation or correlation interaction therefore; then setting first parameters that define a carrier waveform, wherein said carrier waveform exhibits an infraslow or slow selected waveform frequency; then setting second parameters that define a high frequency waveform, that is nested upon said said carrier waveform, and wherein at least one of said carrier waveform, and said high frequency waveform are defined to correspond to physiologic neural oscillations or firing modes associated with at least one of said identified networks or interacting brain regions of interest; then providing one or more implantable pulse generators configured to generate a plurality of nested stimulation via electrical, magnetic, sound, or optic waveforms defined to reinstate one or more desired normal correlation interactions or normal anticorrelation interactions, or normal uncorrelated interactions in said patient; and then reinstating normal anticorrelated, normal correlated interactions or normal uncorrelated interactions by delivering a first nested stimulation waveform through one or more electrodes of said implantable pulse generators to one or more other identified regions of interacting brain regions within said networks of interest. Said normalization of interactions may consist of delivering an infraslow or a slow wave that is in phase, between said nodes or said brain regions of said identified networks. In this method, disruption of correlated or anticorrelated interactions between said nodes within identified networks results in uncorrelated activity within the identified networks and consists of pseudorandom or random delivery of the phases of infraslow or of slow carrier waves thereto. Said networks in which said phase of said infraslow or said slow carrier wave in different regions are used treated to correct an abnormal correlated, anticorrelated or uncorrelated condition or conditions constitute at least one network selected from the group consisting of the default mode, salience, central executive, emotional, affective, somatosensory, medial, lateral and descending pain, auditory, vestibular, visual, olfactory, gustatory, social, mirror neuron, dorsal attention, ventral attention, reward, dysreward, central sympathetic, or parasympathetic networks. This method may comprise the additional step of delivering at least a second or more nested stimulation waveforms through one or more electrodes of said implantable pulse generators to a second or more other identified networks of interacting brain regions of interest. This method may further comprise the additional step of delivering at least a third or more nested stimulation waveforms through one or more electrodes of said implantable pulse generators to a third or more other identified networks of interacting brain regions of interest.

In another alternative preferred embodiment of the invention, there is a method of treating a central nervous system disorder in a patient in need thereof, comprising the steps of identifying abnormal communication connectivity between at least two or more identified networks of interacting brain regions of interest where said communication connectivity has been disrupted from a normal level of communication activity and said disruption is related to a central nervous system related disorder; then determining whether said disrupted communication connectivities are an abnormal communication connectivity correlation interaction or an abnormal communication connectivity anticorrelation interaction between a first identified network of interacting brain regions of interest with one or more other identified networks of interacting brain regions of interest, then setting first parameters that define a carrier waveform, wherein said carrier waveform exhibits an infraslow or slow selected waveform frequency in a range of frequencies of up to 1 Hz; then setting second parameters that define a high frequency waveform, that is nested upon said carrier waveform, and wherein at least one of said carrier waveform and said high frequency waveform are defined to correspond to physiologic neural oscillations associated with at least one of said networks of interacting brain regions of interest; then providing and operating at least one implantable pulse generator configured so as to generate a plurality of said nested stimulation electrical waveforms, which individually are comprised of said carrier waveform and of said high frequency waveform, and are configured to reinstate one or more desired normal communication connectivity correlation interactions or normal communication connectivity anticorrelation interactions in said patient; followed by reinstating normal anticorrelated or normal correlated communication connectivity interactions by delivering a first nested stimulation waveform through one or more applied electrodes of said pulse generator to a first network of interacting brain regions of interest and by delivering a second nested stimulation waveform through one or more applied electrodes of said pulse generator to an other network of interacting brain regions of interest; and then ascertaining whether said patient is in need of a third or more other nested stimulation waveforms selected from the group consisting of a noise waveform, a burstwaveform, or a tonic waveform, delivering a third or more nested stimulation waveforms through one or more applied electrodes of said pulse generator to a third or more additional other networks of interacting brain regions of interest.

In an alternative preferred embodiment of the implantable neurostimulator device of the present invention, for treating a neurological disorder in a patient in need thereof, the device is so configured as to execute the steps of determining the presence of an abnormal correlation interaction or an abnormal anticorrelation interaction between at least one or more nodes of a first identified network of interacting brain regions of interest with at least one or more nodes of one or more other identified networks of interacting brain regions of interest, and determining whether to reinstate a desired normal anticorrelation interaction therefore or a desired normal correlation interaction therefore; then setting first parameters that define a carrier waveform, wherein said carrier waveform exhibits an infraslow selected waveform frequency; then setting second parameters that define a high frequency waveform, that is nested upon said carrier waveform, and wherein at least one of said carrier waveform and said high frequency waveform are defined to correspond to physiologic neural oscillations associated with at least one of said identified networks of interacting brain regions of interest; then providing one or more implantable pulse generators configured to generate a plurality of nested stimulation electrical waveforms defined to reinstate one or more desired normal correlation interactions or normal anticorrelation interactions in said patient; then reinstating normal anticorrelated or normal correlated interactions by delivering a first nested stimulation waveform through one or more electrodes of said pulse generators to said first identified network of interacting brain regions of interest; and finally delivering a second nested stimulation waveform through one or more electrodes of said implantable pulse generators to said one or more other identified networks of interacting brain regions of interest. This embodiment of the implantable neurostimulator device produces a carrier waveform that exhibits a slow selected waveform frequency.

In another alternative preferred embodiment of the method of the invention of achieving central nervous system network normalization, the steps are carried out of firstly determining the presence of an abnormal correlation interaction, an abnormal anticorrelation interaction, or an abnormal uncorrelated interaction between two or more nodes within an identified brain network of interest or between two or more identified brain networks of interest, then determining whether to reinstate a desireable central nervous system network normalization, then setting first parameters that define a carrier waveform; then setting second parameters that define a nested waveform that is nested upon said carrier waveform, and wherein at least one of said carrier waveform and said nested waveform are defined to correspond to physiologic neural oscillations or firing modes associated with at least one of said identified networks; then providing one or more implantable pulse generators configured to generate a plurality of nested stimulations via electrical, magnetic, sound, or optic waveforms defined to reinstate one or more desired normal correlation interactions or normal anticorrelation interactions or normal uncorrelated interactions in said patient; and then proceeding on to reinstating the desireable central nervous system network normalization by delivering a first nested stimulation waveform through one or more electrodes of said implantable pulse generator to said identified brain region or regions of interest within a network of interest and by delivering a second nested stimulation waveform through one or more electrodes of said implantable pulse generator to one or more other identified interacting brain regions within said network of interest. In this method, said desireable network normalization comprises normalization of intra-network or inter-network communication connectivity; and furthermore, wherein said central nervous system desireable network normalization is achieved within a single identified central nervous system network, or where desireable network normalization is achieved between two or more identified central nervous system networks. The method can achieve normalization of phase anticorrelation interactions, by virtue of a carrier waveform that exhibits an infraslow frequency of about 0.1 Hz or less or that exhibits a slow frequency of from about 0.1 to about 1.0 Hz. Furthermore, this method may achieve the normalization of phase correlation interactions by virtue of a carrier waveform that exhibits an infraslow frequency of 0.1 Hz or less, or that exhibits a slow frequency of 0.1 to 1.0 Hz. Alternatively, said method achieves the normalization of uncorrelated phase interactions by virtue of an infraslow frequency of 0.1 Hz or less.\or of a slow frequency of 0.1 to 1.0 Hz. In this method, said identified network of interest is selected from the group consisting of the Central Executive Network, the Salience Network, the Default Mode Network, the Emotional Network, the Mirror Neuron Network, the Ventral Attention Network, or the Dorsal Attention Network. In the practice of this embodiment of the invention, the identified network may constitute a triple network, which may in turn be comprised of the Central Executive Network, the Salience Network, and the Default Mode Network. In this embodiment of the method, said phase anticorrelation interaction nested electrical waveform is a waveform selected from the group consisting of no-stimulation, noise, burst, tonic, a combination of noise and burst, a combination of noise and tonic, or a combination of burst and tonic, while said phase correlation interaction nested electrical waveform is a waveform type selected from the group consisting of no-stimulation, noise, burst, tonic, a combination of noise and burst, a combination of noise and tonic, or a combination of burst and tonic, and wherein said phase uncorrelated interaction nested electrical waveform is a waveform type selected from the group consisting of no-stimulation, noise, burst, tonic, a combination of noise and burst, a combination of noise and tonic, or a combination of burst and tonic waveform. Said noise waveform may be a full band waveform, or a narrow band waveform. Where it is a full band waveform, it may comprise a color of noise selected from pink, blue, brown, red, gray, black, white, violet or green noise, and where it is a narrow band waveform, it may be the noise known as white noise, that is comprised of all frequencies of equal signal strength, or it may be a color of noise of frequency 1/f^{α}, where α is an integer of from −5 to +5. This method may treat disease states of the central nervous system including a disease selected from the group comprising neurological diseases, psychiatric diseases, central nervous system-mediated immunological dysfunctions, or central nervous system-mediated endocrine dysfunctions. Finally, in this method, said network of interest is selected from the group consisting of the emotional/affective, somatosensory, medial, lateral and descending pain, auditory, vestibular, visual, olfactory, gustatory, social networks, mirror neuron, dorsal attention, ventral attention, reward, dysreward, central sympathetic, or central parasympathetic network.

It is to be understood that the subject matter described herein is not limited in its application to the details of patient diagnosis and the design of therapeutic intervention for that patient as set forth in the described disclosure herein or illustrated in the drawings hereof. The subject matter described herein is capable of other methodological embodiments and of being practiced or of being carried out in various ways. Also, it is to be understood that the phraseology and terminology used herein is for the purpose of description and should not be regarded as limiting. The use of “including,” “comprising,” or “having” and variations thereof herein is meant to encompass the anatomy and functionality of central nervous system networks and nodes, the histological tissues, and items listed thereafter and equivalents thereof as well as additional items. Furthermore, it is to be understood that the above description is intended to be illustrative, and not restrictive. For example, the above-described embodiments (and/or aspects thereof) may be applied in combination with each other. In addition, many modifications may be made to adapt a particular situation or therapeutic design to the teachings of the invention without departing from its scope. While the types of apparatus described herein are intended to define the electric signal or current output neuromodulatory parameters of the invention, they and their operational output are by no means limiting and are exemplary embodiments. Many other embodiments will be apparent to those of skill in the art after reviewing and learning the above described disclosure. The scope of the invention should, therefore, be determined with reference to the appended claims, along with the full scope of equivalents to which such claims are entitled. In the appended claims, the terms “including” and “in which” may be used as the plain-English equivalents of the respective terms “comprising” and “wherein.” Moreover, in the following claims, the terms “first,” “second,” and “third,” etc. are used merely as labels, and are not intended to impose numerical requirements on their objects. Further, the limitations of the following claims are not written in means-plus-function format and are not intended to be interpreted based on 45 U.S.C. § 112(f), unless and until such claim limitations expressly use the phrase “means for” followed by a statement of function void of further structure.

What is claimed is:

1. A method of treating a central nervous system disorder in a patient in need thereof, comprising the steps of:
(a) determining the presence of an abnormal correlation interaction or an abnormal anticorrelation interaction or an abnormal uncorrelated interaction between at least one or more nodes within an identified brain network of interest and determining whether to reinstate a desired normal anticorrelation therefore or a desired normal correlation interaction therefore;
(b) setting first parameters that define a carrier waveform, wherein said carrier waveform exhibits an infraslow or a slow selected waveform frequency;
(c) setting second parameters that define a high frequency waveform, that is nested upon said carrier waveform, and wherein at least one of said carrier waveform, and said high frequency waveform are defined to correspond to physiologic neural oscillations or firing modes associated with at least one of said identified networks or interacting brain regions of interest;
(d) providing one or more implantable pulse generators configured to generate a plurality of nested stimulation waveforms defined to reinstate one or more desired normal correlation interactions or normal anticorrelation interactions, in said patient; and
(e) reinstating normal anticorrelated or normal correlated interactions by delivering a first nested stimulation waveform through one or more electrodes of said implantable pulse generators to one or more other identified regions of interacting brain regions within said network of interest; and
(f) delivering a second nested stimulation waveform through one or more electrodes of said pulse generators to said one or more other identified networks of interacting brain regions of interest.

2. The method as claimed in claim 1, comprising the additional step of delivering at least a third or more nested stimulation waveforms through one or more electrodes of said pulse generators to a third or more other identified networks of interacting brain regions of interest.

3. The method as claimed in claim 1, additionally including the step of reinstating a normal uncorrelated interaction.

4. The method as claimed in claim 1, wherein said normalization of an interaction consists of delivering an infraslow or a slow wave that is in phase, between said nodes or said brain regions of said identified network.

5. The method as claimed in claim 1, characterized in that disruption of correlated or anticorrelated interactions between said nodes within an identified network results in uncorrelated activity within the identified network and consists of pseudorandom or random delivery of the phases of infraslow or of slow carrier waves.

6. The method as claimed in claim 1, wherein said networks in which said phase of said infraslow or said slow carrier wave in different regions are used to treat or correct one or more abnormal correlated, anticorrelated or uncorrelated conditions constitute a network selected from the group consisting of the default mode, salience, central executive, emotional, affective, somatosensory, medial, lateral and descending pain, auditory, vestibular, visual, olfactory, gustatory, social, mirror neuron, dorsal attention, ventral attention, reward, dysreward, central sympathetic, or parasympathetic networks.

7. The method as claimed in claim 1, for treatment of nervous system disorders, for neuroenhancement, for ameliorating non-pathological states, or for strengthening neurological resilience by:
(a) comprising the additional step of ameliorating one or more abnormal interactions between said first brain network and at least one or more further brain networks
(b) delivering a first infraslow stimulation wave of from about 0.01 Hz to about 0.1 Hz, or a first slow stimulation wave of from about 0.1 Hz to about 1 Hz) stimulation wave to said first brain network; and
(c) delivering a second infraslow stimulation wave of from about 0.01 Hz to about 0.1 Hz, or a second slow stimulation wave of from about 0.1 Hz to about 1 Hz) stimulation wave to said second brain network;
wherein a phase difference between said first and said second infraslow or said first and said second slow stimulation waves is selected for amelioration of said abnormal interaction.

8. The method as claimed in claim 1, for treatment of nervous system disorders, for neuroenhancement, for ameliorating non-pathological states, or for strengthening neurological resilience by comprising the additional step of ameliorating an abnormal condition within a first brain network and ameliorating one or more abnormal interactions between said first brain network and at least one or more further brain networks by:
(a) delivering a first infraslow stimulation wave of from about 0.01 Hz to about 0.1 Hz, or a first slow stimulation wave of from about 0.1 Hz to about 1 Hz) stimulation wave to said first brain network; and
(b) delivering a second infraslow stimulation wave of from about 0.01 Hz to about 0.1 Hz, or a second slow stimulation wave of from about 0.1 Hz to about 1 Hz) stimulation wave to said second brain network; and
wherein a phase difference between said first and said second infraslow or said first and said second slow stimulation waves is selected for amelioration of the abnormal interaction.

9. A method of treating a central nervous system disorder in a patient, comprising the steps of:
(a) identifying abnormal connectivity between at least two or more identified networks of interacting brain regions where connectivity has been disrupted from its normal communication activity and said disruption is related to a central nervous system disorder;
(b) recording electrical activity related to neural activity in the brain of said patient;
(c) with reference to said recording, determining the presence of an abnormal correlation interaction or an abnormal anticorrelation interaction between each identified network of interacting brain regions with each of the remaining other identified networks of interacting brain regions, and determining whether to reinstate a normal anticorrelated or a normal correlated interaction;
(d) setting first parameters that define a stimulation carrier waveform, wherein said carrier waveform exhibits an infraslow or slow selected waveform frequency in a range of frequencies of up to 1 Hz;
(e) setting second parameters that define a stimulation high frequency waveform, where said high frequency waveform is nested upon said carrier waveform, and wherein at least one of said stimulation carrier waveform and said high frequency waveform are defined to correspond to physiologic neural oscillations associated with at least one of said networks of interacting brain regions;
(f) providing and operating an implantable pulse generator to generate a plurality of nested stimulation electrical waveforms, which individually are comprised of said carrier waveforms and of said high frequency waveforms, wherein said nested stimulation waveform comprises a plurality of pulse bursts, and wherein said pulse bursts are characterized in that each of said pulse bursts is comprised of a plurality of discrete pulses, and said plurality of discrete pulses within each pulse burst are repeated according to a frequency parameter of said high frequency waveform, and wave amplitude of each said discrete pulse within respective said pulse bursts is controlled according to said high frequency waveform nested on said carrier waveform such that wave amplitude peaks of the corresponding plurality of said discrete pulses vary within each respective said pulse burst;
(g) reinstating normal anticorrelated or normal correlated interactions by delivering a first nested stimulation waveform through one or more applied implantable pulse generator electrodes to a first network of interacting brain regions of interest, and delivering a second nested stimulation waveform through one or more applied pulse generator electrodes to a second network of interacting brain regions of interest; and
(h) where said patient is in need of a third or more other nested stimulation waveforms, delivering a third or more nested stimulation waveforms through one or more applied electrodes of said pulse generator to a third or more additional other networks of interacting brain regions of interest.

10. The method as claimed in claim 9, wherein one or more of said nested stimulation electrical waveforms is selected from the group consisting of pink noise, brown noise, red noise, black noise, grey noise, white noise, blue noise, violet noise, or green noise.

11. The method as claimed in claim 9, wherein one or more of said nested stimulation electrical waveforms is defined as 1/f^{α}, with f being wave frequency and α being any number between −5 and +5 or is defined as a composite of 1/f^{α} and 1/f^{-α}, with f being wave frequency and α being any number between −5 and +5.

12. The method as claimed in claim 9, where said nested noise stimulation electrical waveforms are generated in a pseudo-random manner.

13. The method as claimed in claim 9 wherein one or more of said nested noise stimulation waveforms comprises pink noise nested on a carrier wave form.

14. The method as claimed in claim 9, wherein one or more of said nested noise stimulation waveforms comprises grey noise nested on a carrier wave form.

15. The method as claimed in claim 9, wherein said networks of interacting brain regions are selected from the central executive network, the default node network, or the salience network.

16. The method as claimed in claim 15, wherein said networks of interacting brain regions are selected from the left and/or right central executive network, the left and/or right default node network, or the left and/or right salience network.

17. The method as claimed in claim 9, wherein said networks additionally comprise a fourth network, selected from the group consisting of the sensorimotor system, the auditory system, the visual system, the somatosensory system, or the motor network.

18. The method as claimed in claim 9, where said networks additionally comprise a fourth network, selected from the group consisting of the attentional network, the dorsal attentional network or the ventral attentional network.

19. The method as claimed in claim 9, where said networks additionally comprise a fourth network, the mirror neuron network.

20. The method as claimed in claim 9, where said networks additionally comprise a fourth network, the affective/emotional network.

21. The method as claimed in claim 9 where said networks additionally comprise a fourth network, the central autonomic network.

22. The method as claimed in claim 9, wherein said networks additionally comprise a fifth networks, selected from the group consisting of the sensorimotor network, one or more of the attentional networks, the mirror neuron network, the emotional/affective network, or the central autonomic network

23. The method as claimed in claim 9, wherein said abnormal interaction between nodes clinically manifests itself as a disease selected from the group consisting of attention deficit hyperactivity disorder, anxiety, depression, bipolar disorder, autism, obsessive compulsive disorder, post-traumatic stress disorder syndrome, or schizophrenia, mild cognitive impairment, Alzheimers dementia, Lewy-body disease dementia, multi-infarct dementia, thalamocortical dysrhythmias, tinnitus, pain, Parkinson Disease, stress, epilepsy, a disorder of consciousness, a minimally cognitive state, a vegetative state, unresponsive wakefulness syndrome, a stress autonomic nervous system disorder or an immune disorders.

24. The method as claimed in claim 9, wherein said abnormal connectivity takes place in communications between said default mode network and said central executive network.

25. The method as claimed in claim 9, wherein said abnormal connectivity communication is reinstated to a normal anticorrelated communication.

26. The method as claimed in claim 9, wherein said abnormal connectivity communication is reinstated to a normal correlated communication.

27. The method as claimed in claim 9, wherein said abnormal connectivity takes place in communications between said central executive network and said salience network.

28. The method as claimed in claim 9, wherein said abnormal connectivity communicates between said salience network and said default mode network.

29. The method as claimed in claim 9, wherein said first, second, and third nested stimulation waveforms are delivered to said patient contemporaneously.

30. The method as claimed in claim 9, wherein said abnormal connectivity communication is reinstated to a normal anticorrelated communication by configuring said infraslow carrier wave form to be opposite in phase between said default mode network and said central executive network.

31. The method as claimed in claim 9, wherein said abnormal connectivity communication is reinstated to a normal anticorrelated communication by configuring said infraslow carrier wave form to be opposite in phase between said default mode network and said salience network.

32. The method as claimed in claim 9, wherein said abnormal connectivity communication is reinstated to a normal anticorrelated communication by configuring said infraslow carrier wave form to be opposite in phase between said central executive network and said salience network.

33. The method as claimed in claim 9, wherein said abnormal connectivity communication is reinstated to a normal anticorrelated communication by configuring said infraslow carrier wave form to be in phase between said default mode network and said central executive network.

34. The method as claimed in claim 12, wherein said abnormal connectivity communication is reinstated to a normal anticorrelated communication by configuring said infraslow carrier wave form to be in phase between said default mode network and said salience network.

35. The method as claimed in claim 12, wherein said abnormal connectivity communication is reinstated to a normal anticorrelated communication by configuring said infraslow carrier wave form to be in phase between said central executive network and said salience network.
